(19) United States
(12) Patent Application Publication
(10) Pub. No.: US 2026/0053499 A1
(43) Pub. Date: Feb. 26, 2026

(54) END TOOL FOR SURGICAL INSTRUMENT

(71) Applicant: LIVSMED INC., Seongnam-si (KR)

(72) Inventor: Woo Jung CHOI, Siheung-si (KR)

(21) Appl. No.: 19/305,070

(22) Filed: Aug. 20, 2025

(30) Foreign Application Priority Data
Aug. 20, 2024 (KR) 10-2024-0111494

(51) Int. Cl. A61B 17/072; A61B 17/29; A61B 34/00

(52) U.S. Cl. CPC A61B 17/07207; A61B 34/71; A61B 2017/2903; A61B 2017/2932

(57) ABSTRACT

Provided is an end tool for a surgical instrument, including one or more jaws that are rotatable, a wire unit including a first wire and a second wire configured to transmit a driving force to the one or more jaws, and a pulley unit positioned to allow at least a portion of the wire unit to come into contact therewith and configured to guide a traveling path of the wire unit, wherein the pulley unit includes at least one double-groove pulley including a first pulley part having a first groove and a second pulley part having a second groove, the second groove being configured to be spaced apart from the first groove, and the first pulley part and the second pulley part are integrally formed.

### CROSS-REFERENCE TO RELATED APPLICATION

This application is based on and claims priority under 35 USC § 119 to Korean Patent Application No. 10-2024-0111494, filed on Aug. 20, 2024, in the Korean Intellectual Property Office, the disclosure of which is incorporated by reference herein in its entirety.

### BACKGROUND

### 1. Field

The present disclosure relates to an end tool for a surgical instrument.

### 2. Description of the Related Art

Medically, surgery refers to the treatment of diseases by cutting, slitting, or manipulating the skin, mucous membranes, or other tissues using medical devices. In particular, open surgery, which cuts and opens the skin of a surgical site and cures, shapes, or removes an organ therein, may cause bleeding, side effects, patient pain, scars, or the like. Accordingly, recently, surgery performed by inserting only a medical device, for example, laparoscopic surgical instrument, microsurgical microscope, and the like by forming a predetermined hole in the skin or surgery using a robot has been spotlighted as an alternative.

A surgical instrument is a tool equipped with an end tool provided on one end of a shaft that passes through a hole drilled in the skin, and is manipulated by a medical doctor by hand using a predetermined driving part or by a robot arm to perform surgery at the surgical site. The end tool provided on the surgical instrument performs a rotational motion, a gripping motion, a cutting motion, or the like through a predetermined structure.

The background art described above is technical information retained by the present inventors in order to derive the present disclosure or obtained by the present inventors in the process of deriving the present disclosure, and thus is not necessarily known art disclosed to the general public before the filing of the present application.

### SUMMARY

The present disclosure is directed to providing an end tool for a surgical instrument that is mountable on a robot arm or operable manually for use in laparoscopic surgery or various other surgical procedures, wherein the end tool is configured to minimize rotational resistance of pulleys and increase space utilization of components.

According to an aspect of the present disclosure, there is provided an end tool for a surgical instrument, the end tool including one or more jaws that are rotatable, a wire unit including a first wire and a second wire configured to transmit a driving force to the one or more jaws, and a pulley unit positioned to allow at least a portion of the wire unit to come into contact therewith and configured to guide a traveling path of the wire unit, wherein the pulley unit includes at least one double-groove pulley including a first pulley part having a first groove and a second pulley part having a second groove, the second groove being configured to be spaced apart from the first groove, and the first pulley part and the second pulley part are integrally formed.

In an embodiment of the present disclosure, the first wire may be wound around the first groove, and the second wire may be wound around the second groove.

In an embodiment of the present disclosure, with respect to a rotational shaft of the at least one double-groove pulley, the first wire may be positioned on one side of the at least one double-groove pulley, and the second wire may be positioned on another side of the at least one double-groove pulley.

In an embodiment of the present disclosure, when the at least one double-groove pulley may rotate in one direction, the first wire and the second wire, which are wound around the at least one double-groove pulley, may move in different directions.

In an embodiment of the present disclosure, the first wire and the second wire may be positioned together on one side of the at least one double-groove pulley with respect to a rotational shaft of the at least one double-groove pulley.

In an embodiment of the present disclosure, when the at least one double-groove pulley rotates in one direction, the first wire and the second wire, which are wound around the at least one double-groove pulley, may move in a same direction.

In an embodiment of the present disclosure, a width of the first groove and a width of the second groove, which correspond to a direction parallel to a rotational shaft of the at least one double-groove pulley, may be different from each other.

In an embodiment of the present disclosure, a diameter of the first pulley part and a diameter of the second pulley part may be different from each other.

In an embodiment of the present disclosure, a pair of wires may be wound around the at least one double-groove pulley, and the pair of wires may control one degree of freedom of the one or more jaws.

In an embodiment of the present disclosure, the one or more jaws may include a first jaw configured to accommodate at least one region of an operation member movable in at least one direction, and a second jaw facing the first jaw and configured to rotate relative to the first jaw, and the first wire and the second wire may be connected to the operation member to enable the operation member to move in at least one direction.

In an embodiment of the present disclosure, the first wire may be a firing wire that moves the operation member toward a distal end of the end tool, and the second wire is a retraction wire that moves the operation member toward a proximal end of the end tool.

In an embodiment of the present disclosure, the pulley unit may further include a first double-groove pulley and a second double-groove pulley configured to be rotatable around a first rotational shaft, the wire unit may further include a third wire and a fourth wire connected to the operation member, the first wire and the second wire may be wound around the first double-groove pulley, and the third wire and the fourth wire may be wound around the second double-groove pulley.

In an embodiment of the present disclosure, the first wire and the third wire may be firing wires that move the operation member toward a distal end of the end tool, and the second wire and the fourth wire may be retraction wires that move the operation member toward a proximal end of the end tool.

In an embodiment of the present disclosure, the pulley unit may further include a first double-groove pulley and a second double-groove pulley configured to be rotatable around a first rotational shaft, the wire unit may further include a third wire and a fourth wire configured to transmit a driving force to the one or more jaws, the first wire and the second wire may be wound around the first double-groove pulley, and the third wire and the fourth wire may be wound around the second double-groove pulley.

In an embodiment of the present disclosure, the pulley unit may further include a third double-groove pulley and a fourth double-groove pulley configured to be rotatable around a third rotational shaft that forms a certain angle with the first rotational shaft, and a fifth double-groove pulley and a sixth double-groove pulley configured to rotate around a fourth rotational shaft disposed parallel to the third rotational shaft.

In an embodiment of the present disclosure, the first wire and the second wire may sequentially come into contact with the first double-groove pulley, the third double-groove pulley, and the fifth double-groove pulley while moving from a distal end toward a proximal end of the end tool, and the third wire and the fourth wire may sequentially come into contact with the second double-groove pulley, the fourth double-groove pulley, and the sixth double-groove pulley.

In an embodiment of the present disclosure, with respect to the first rotational shaft, the first wire and the fourth wire may be positioned on one side of the first double-groove pulley and one side of the second double-groove pulley, respectively, and the second wire and the third wire may be positioned on another side of the first double-groove pulley and another side of the second double-groove pulley, respectively.

According to another aspect of the present disclosure, there is provided a double-groove pulley applicable to a surgical instrument, the double-groove pulley including a first pulley part including a first groove around which a first wire is wound, and a second pulley part including a second groove, which is configured to be spaced apart from the first groove and around which a second wire is wound, wherein the first pulley part and the second pulley part are integrally formed.

In an embodiment of the present disclosure, the first wire and the second wire may control one degree of freedom of the surgical instrument.

In an embodiment of the present disclosure, with respect to a rotational shaft of the double-groove pulley, the first wire may be positioned on one side of the double-groove pulley, and the second wire may be positioned on another side of the double-groove pulley.

In an embodiment of the present disclosure, when the double-groove pulley may rotate in one direction, the first wire and the second wire, which are wound around the double-groove pulley, may move in different directions.

In an embodiment of the present disclosure, the first wire and the second wire may be positioned together on one side of the double-groove pulley with respect to a rotational shaft of the double-groove pulley.

In an embodiment of the present disclosure, when the double-groove pulley rotates in one direction, the first wire and the second wire, which are wound around the double-groove pulley, may move in a same direction.

In an embodiment of the present disclosure, a width of the first groove and a width of the second groove, which correspond to a direction parallel to a rotational shaft of the double-groove pulley, may be different from each other.

In an embodiment of the present disclosure, a diameter of the first pulley part and a diameter of the second pulley part may be different from each other.

Other aspects, features, and advantages other than those described above will become apparent from the following drawings, claims, and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings in which:

FIG. 1 is a perspective view illustrating a surgical instrument to which an end tool according to an embodiment of the present disclosure is applied;

FIG. 2 is a perspective view illustrating a reload assembly to which the end tool according to an embodiment of the present disclosure is applied;

FIG. 3 is a perspective view illustrating the end tool according to an embodiment of the present disclosure;

FIG. 4 is a perspective view for describing an end tool hub and a pitch hub of the end tool of FIG. 3;

FIG. 5 is a perspective view for describing a rotational shaft and pulley structure of the end tool of FIG. 3;

FIG. 6 is a perspective view illustrating a double-groove pulley and a rotational shaft according to an embodiment of the present disclosure;

FIG. 7 is a front view illustrating the double-groove pulley according to an embodiment of the present disclosure;

FIG. 8 is a front view illustrating a double-groove pulley according to another embodiment of the present disclosure;

FIG. 9 is a front view illustrating a double-groove pulley according to another embodiment of the present disclosure;

FIG. 10 is a view for describing a relationship between the double-groove pulley and wires according to an embodiment of the present disclosure;

FIG. 11 is a side view schematically illustrating the first double-groove pulley and the wires of the end tool of FIG. 3;

FIG. 12 is a front view of the first double-groove pulley of FIG. 11 as viewed from one direction;

FIG. 13 is a cross-sectional view illustrating a cross section of the first double-groove pulley of FIG. 11;

FIG. 14 is a front view schematically illustrating a first double-groove pulley and a second double-groove pulley, and wires of the end tool of FIG. 3;

FIG. 15 is a cross-sectional view illustrating cross sections of the first double-groove pulley and the second double-groove pulley of FIG. 14;

FIG. 16 is a perspective view schematically illustrating the first and second double-groove pulleys and the wires of the end tool of FIG. 3;

FIG. 17 is a perspective view illustrating a state in which an end tool hub of the end tool of FIG. 3 is removed;

FIGS. 18 and 19 are perspective views illustrating a state in which a jaw pulley is removed from the end tool of FIG. 17;

FIG. 20 is a plan view for describing paths of wires wound around the first double-groove pulley in the end tool of FIG. 3;

FIG. 21 is a perspective view of the end tool of FIG. 20 viewed from another direction;

FIG. 22 is a plan view for describing paths of wires wound around the second double-groove pulley in the end tool of FIG. 3;

FIG. 23 is a perspective view of the end tool of FIG. 22 viewed from another direction;

FIG. 24 is a perspective view illustrating a surgical instrument to which an end tool according to another embodiment of the present disclosure is applied;

FIG. 25 is a schematic perspective view for describing the end tool of FIG. 24;

FIG. 26 is a perspective view of the end tool of FIG. 25 viewed from another direction;

FIG. 27 is a schematic perspective view of the end tool of FIG. 25 with a second jaw removed;

FIG. 28 is a schematic perspective view of the end tool of FIG. 27 with a cartridge removed;

FIG. 29 is a transparent perspective view of FIG. 28; and

FIG. 30 is a perspective view illustrating a first jaw and the cartridge of the surgical instrument of FIG. 24.

### DETAILED DESCRIPTION

Hereinafter, the following embodiments will be described in detail with reference to the accompanying drawings. When describing with reference to the drawings, identical or corresponding components will be assigned the same reference numerals and duplicate descriptions thereof will be omitted.

Since various transformations can be made to these embodiments, specific embodiments will be illustrated in the drawings and described in detail in the detailed description. The effects and features of the present embodiments and the accompanying methods thereof will become apparent from the following description of the contents, taken in conjunction with the accompanying drawings. However, the present embodiments are not limited to the embodiments disclosed below, but may be implemented in various forms.

In describing the present disclosure, a detailed description of known related arts will be omitted when it is determined that the gist of the present disclosure may be unnecessarily obscured.

In the following embodiments, singular forms are intended to include plural forms as well, unless the context clearly indicates otherwise. Although terms such as “first,” “second,” and the like may be used to describe various components, such components should not be limited to the above terms. The terms are only used to distinguish one component from another.

In the following embodiments, terms such as “include” or “have” means that the features or components described in the specification are present, and the possibility that one or more other features or components will be added is not excluded in advance.

In the following embodiments, when a unit, region, or component is referred to as being formed on another unit, region, or component, it can be directly formed on the other unit, region, or component. That is, for example, intervening units, regions, or components may be present.

In the following embodiments, terms such as “connecting” or “coupling” two members do not necessarily mean a direct and/or fixed connection or coupling of the two members, unless the context clearly indicates otherwise, and do not preclude another members from being interposed between the two members.

Sizes of components in the drawings may be exaggerated or reduced for convenience of description. For example, since the size and thickness of each component shown in the drawings are arbitrarily illustrated for convenience of description, the following embodiments are not necessarily limited thereto.

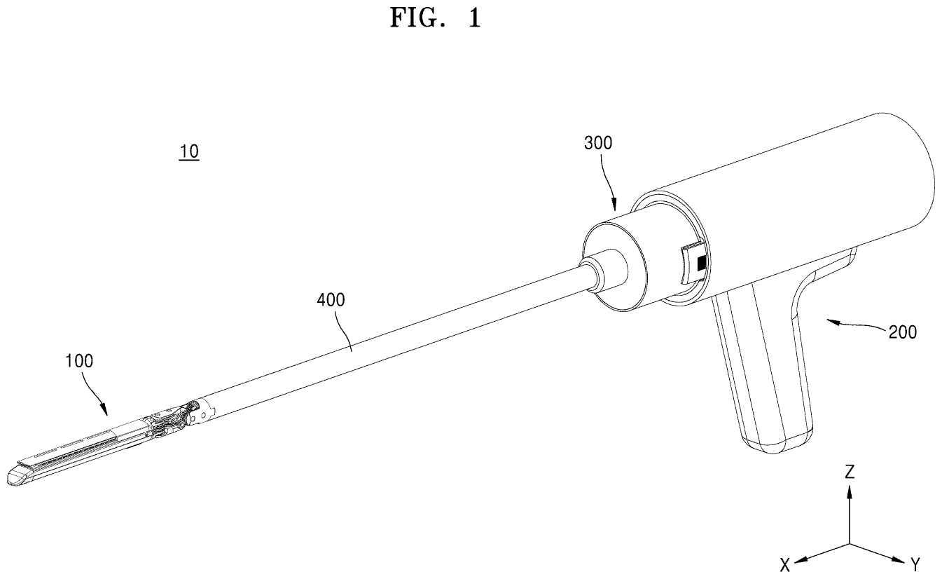

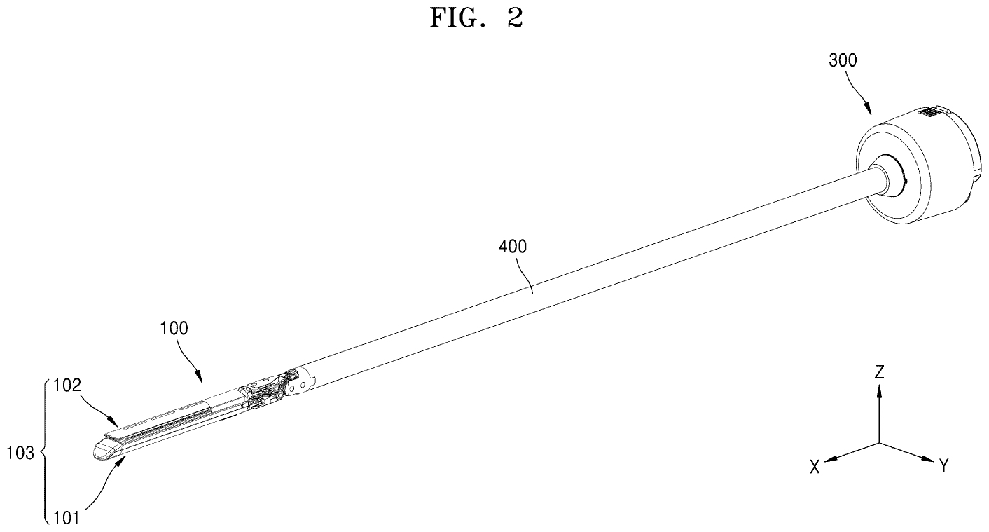

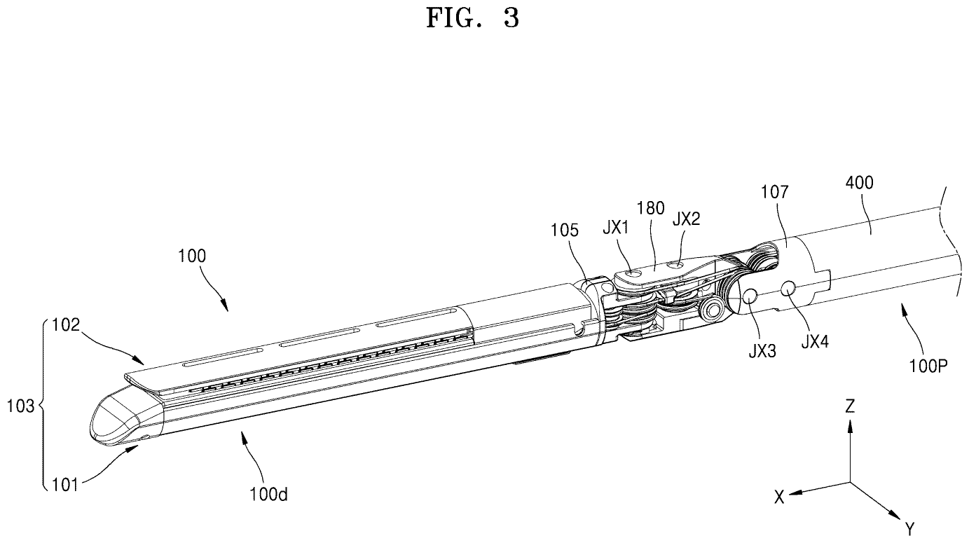

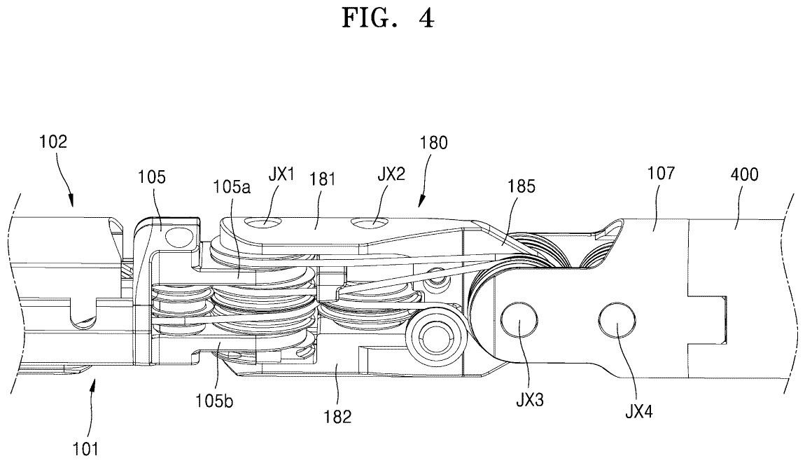

FIG. 1 is a perspective view illustrating a surgical instrument to which an end tool according to an embodiment of the present disclosure is applied, and FIG. 2 is a perspective view illustrating a reload assembly to which the end tool according to an embodiment of the present disclosure is applied. FIG. 3 is a perspective view illustrating the end tool according to an embodiment of the present disclosure. FIG. 4 is a perspective view for describing an end tool hub and a pitch hub of the end tool of FIG. 3.

In describing the present disclosure, the portion closer to a user side, i.e., the portion closer to a manipulation part 200, will be referred to as a proximal end, and the portion farther from the user side, that is, the portion closer to an end portion of an end tool 100, will be referred to as a distal end.

For example, the portion of the end tool 100 closer to a connection part (or shaft) 400 will be defined as a proximal end 100p, and the portion farther from the connection part (or shaft) 400, that is, the portion closer to the end portion of the end tool 100 will be defined and described as a distal end 100d.

The end tool 100 according to an embodiment of the present disclosure may be connected to one end portion of the connection part (shaft) of a surgical instrument 10 and inserted into a surgical site to perform motions required for surgery.

First, the surgical instrument and the reload assembly, to which the end tool 100 according to an embodiment of the present disclosure is applicable, will be described.

Referring to FIGS. 1 to 3, the surgical instrument according to the present embodiment may include the end tool 100, the manipulation part 200, the connection part 400, and a power transmission part 300.

In some embodiments, as shown in FIG. 2, a configuration including the end tool 100, the connection part (or shaft) 400, and the power transmission part 300 will be distinguished and referred to as the reload assembly.

In some embodiments, the connection part 400 is formed in the shape of a hollow shaft, in which one or more wires and electric wires may be accommodated. For convenience of description of the present embodiment, the shaft will be referred to and described as the connection part 400. The manipulation part 200 is coupled to one end portion of the connection part 400, the end tool 100 is coupled to another end portion thereof, and the connection part 400 may serve to connect the manipulation part 200 to the end tool 100. As an example, the connection part 400 may include a straight part, and although not shown in the drawings, the connection part 400 may include one or more curved parts to increase ease of use and control the arrangement of components for manipulation.

The power transmission part 300 may be formed on one end portion of the connection part 400 and may serve to transmit power generated from a power generation part to be described later to the end tool 100. For example, the power transmission part 300 may be positioned between the end tool 100 and the manipulation part 200. As will be described later, when a user such as a medical doctor manipulates the manipulation part 200, the power generation part generates power to control the end tool 100, and the generated power may be transmitted to the end tool 100 through the power transmission part 300. The power transmission part 300 may include a plurality of wires, a plurality of pulleys, a plurality of links, a plurality of joints, a plurality of gears, and the like.

A user may operate the end tool 100 through the manipulation of the manipulation part 200. For example, the manipulation part 200 is a configuration for the user to input signals to control the motions of the end tool 100. That is, the manipulation part 200 may be said to be a configuration that receives signals from the user to control the operation of the end tool 100. In some embodiments, the signals for controlling the motions of the end tool 100 may correspond to mechanical manipulations such as pressing a button or switch, or rotating or moving a particular member, and may also be electrical signals generated by such mechanical manipulations, but the present disclosure is not limited thereto. The manipulation part 200 is provided as an interface to be directly controlled by a medical doctor, for example, provided in a gun shape, a tongs shape, a stick shape, a lever shape, or the like, and when the medical doctor controls the manipulation part 200, the end tool 100, which is connected to the corresponding interface and inserted into the body of a surgical patient, performs a certain motion, thereby performing surgery. In some embodiments, the manipulation part 200 is illustrated in FIG. 1 as being formed in a gun shape, but the concept of the present disclosure is not limited thereto, and various types of manipulation parts that can be connected to the end tool 100 and manipulate the end tool 100 may be possible.

The manipulation part 200 may include a housing 201 forming an outer shape of the manipulation part 200. As will be described later, at least a portion of the power generation part configured to generate power to control the end tool 100 may be accommodated inside the housing 201. In some embodiments, a circuit unit for controlling the operation of the power generation part and a slip ring for supplying electrical energy to the power generation part, connecting communication, or transmitting various other signals may be accommodated inside the housing 201.

A handle 202 may be formed on the manipulation part 200. The handle 202 is a portion for a user to grip. Thus, the user can use a surgical instrument 10 according to the present disclosure while gripping the handle 202 of the manipulation part 200.

In some embodiments, although not shown in the drawings, a button, a switch, a lever, and the like for controlling various motions of the end tool 100 may be further formed in the manipulation part.

The end tool 100 is formed on another end portion of the connection part 400, and performs necessary motions for surgery by being inserted into a surgical site. As an example of the end tool 100, a pair of jaws 103 for performing a grip motion may be used. However, the concept of the present disclosure is not limited thereto, and various devices for performing surgery may be used as the end tool 100. For example, a configuration such as a cantilever cautery may also be used as the end tool. The end tool 100 is connected to the manipulation part 200 by the power transmission part 300 (e.g., a wire or the like), and receives a driving force of the manipulation part 200 through the power transmission part 300 to perform a motion necessary for surgery, such as gripping, cutting, suturing, or the like.

Hereinafter, the end tool 100 for the surgical instrument of FIG. 3 will be described in more detail.

The end tool 100 according to an embodiment of the present disclosure may include one or more jaws, a wire unit, and a pulley unit.

The jaws may perform various functions, for example, a grip motion, and may include a pair of jaws, e.g., a first jaw 101 and a second jaw 102 as a specific example. In some embodiments, each of the first jaw 101 and the second jaw 102, or a component encompassing the first jaw 101 and the second jaw 102 may be referred to as the jaw 103.

The first jaw 101 and the second jaw 102 may be disposed to face each other, may move closer to or move away from each other, and may be configured to rotationally move around, for example, one shaft.

The first jaw 101 may be partially accommodated in an end tool hub 180 to be described later, and may perform yaw rotation around a first rotational shaft JX1. In some embodiments, the first jaw 101 may perform pitch rotation around a third rotational shaft JX3 to be described later. The first jaw 101 will be described in detail later.

Referring to FIG. 4, the end tool 100 may include one or more members that connect the jaw 103 to the connection part 400, such as a joint member. Further, in an optional embodiment, the end tool 100 may include the end tool hub 180 and a pitch hub 107.

The end tool hub 180 may be disposed to connect the jaw 103 to the connection part 400. In other words, the end tool hub 180 may be a member connecting the connection part 400 to the first jaw 101 and the second jaw 102. Alternatively, the end tool hub 180 may be disposed to connect the jaw 103 to the pitch hub 107. That is, the end tool hub 180 may be a connection member interposed between the jaw 103 and the pitch hub 107.

The end tool hub 180 may be configured to internally accommodate at least a portion of the first jaw 101.

In some embodiments, the pitch hub 107 may be configured to be axially coupled to the end tool hub 180 and to be rotatable relative to the end tool hub 180. In some embodiments, the end tool hub 180 and the pitch hub 107 may be axially coupled through the third rotational shaft JX3. Specific details of the pitch hub 107 will be described later.

The end tool hub 180 may include a pair of jaw pulley coupling parts 181 and 182 and a pitch pulley coupling part 185.

In detail, the pair of jaw pulley coupling parts 181 and 182 are formed to face each other so that a plurality of pulleys can be accommodated therein. In some embodiments, a through hole is formed in each of the jaw pulley coupling parts 181 and 182 so that the first rotational shaft JX1 passes therethrough and axially couple the jaw pulley coupling parts 181 and 182 to the pulleys.

The pair of jaw pulley coupling parts 181 and 182 may be connected to each other by the pitch pulley coupling part 185. That is, the pair of jaw pulley coupling parts 181 and 182 are coupled to each other by the pitch pulley coupling part 185 formed in a direction substantially perpendicular thereto, so that the pair of jaw pulley coupling parts 181 and 182 and the pitch pulley coupling part 185 form a substantially “C” shape, in which a plurality of pulleys are accommodated.

In other words, it may be expressed that the pair of jaw pulley coupling parts 181 and 182 are formed to extend in an X-axis direction from both end portions of the pitch pulley coupling part 185 formed to be elongated in a Z-axis direction.

In some embodiments, a pitch pulley 131 around which a pitch wire may be wound may be coupled to the pitch pulley coupling part 185. Alternatively, the pulley 131 may not rotate around a shaft in the conventional sense of a pulley. Instead, it may be fixed as part of the end tool hub 180 and serve a similar function by having a wire wound around it. In some embodiments, the pitch pulley coupling part 185 may be formed on an XZ plane. In some embodiments, a through hole through which the third rotational shaft JX3 may be inserted may be formed in the pitch pulley coupling part 185.

The first rotational shaft JX1 and a second rotational shaft JX2 may be inserted through the end tool hub 180, and the end tool hub 180 may internally accommodate at least a portion of the pulleys axially coupled to the first rotational shaft JX1. In some embodiments, the end tool hub 180 may internally accommodate at least some of pulleys axially coupled to the second rotational shaft JX2.

In an embodiment, the second rotational shaft JX2 may be positioned adjacent and parallel to the first rotational shaft JX1.

In another embodiment, the second rotational shaft JX2 may be disposed alongside the first rotational shaft JX1 but may not be parallel to the first rotational shaft JX1. That is, the first rotational shaft JX1 is disposed in the Z-axis direction in the drawing, whereas the second rotational shaft JX2 may be disposed obliquely at a predetermined angle with respect to a Z-axis.

In some embodiments, the pitch pulley 131 serving as an end tool pitch pulley may be formed at one end portion of the end tool hub 180. The pitch pulley 131 may be integrally formed with the end tool hub 180 as one body. That is, a disk-shaped pulley is formed at one end portion of the end tool hub 180, and a groove around which a wire may be wound may be formed on an outer circumferential surface of the pulley. Alternatively, the pitch pulley 131 may be formed as a separate member from the end tool hub 180 to be coupled to the end tool hub 180. The pitch wire is coupled to the pitch pulley 131 serving as an end tool pitch pulley, and when the pitch pulley 131 rotates around the third rotational shaft JX3, the end tool hub 180 also rotates together with the pitch pulley 131, thereby performing a pitch motion.

In some embodiments, the pitch hub 107 may be configured to be axially coupled to the end tool hub 180 and to be rotatable relative to the end tool hub 180. In some embodiments, the end tool hub 180 and the pitch hub 107 may be axially coupled to each other through the third rotational shaft JX3.

The pitch hub 107 may have a disk-shaped body on the side coupled to the connection part 400, and may include an extension portion extending toward the end tool hub 180.

In some embodiments, the third rotational shaft JX3 and a fourth rotational shaft JX4, which will be described later, may be inserted through the extension portion extending toward the end tool hub 180, and the pitch hub 107 and the end tool hub 180 (and the pitch pulley 131) may be axially coupled to each other by the third rotational shaft JX3. Thus, the end tool hub 180 and the pitch pulley 131 may be formed to be rotatable around the rotational shaft JX3 with respect to the pitch hub 107.

Further, the pitch hub 107 may internally accommodate at least some of pulleys axially coupled to the third rotational shaft JX3. In some embodiments, the pitch hub 107 may internally accommodate at least some of pulleys axially coupled to the fourth rotational shaft JX4.

In some embodiments, the third rotational shaft JX3 may function as a pitch rotational shaft, and the fourth rotational shaft JX4 may function as an auxiliary pulley rotational shaft.

That is, the auxiliary pulley rotational shaft JX4 may be disposed parallel to and adjacent to the pitch rotational shaft JX3.

In some embodiments, the pitch hub 107 may further include a pitch auxiliary pulley 132 that rotates around the fourth rotational shaft JX4.

Hereinafter, the pulley units axially coupled to the respective rotational shafts will be described in detail.

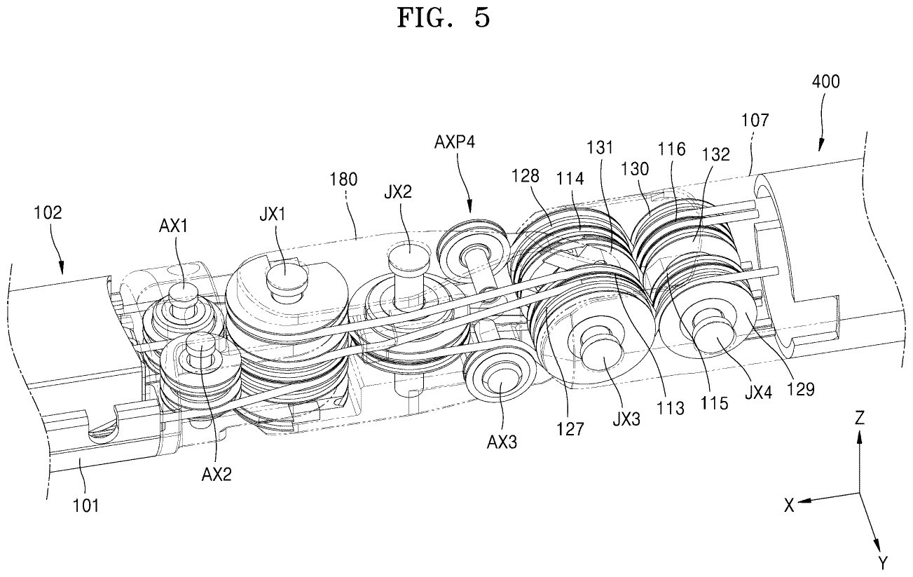

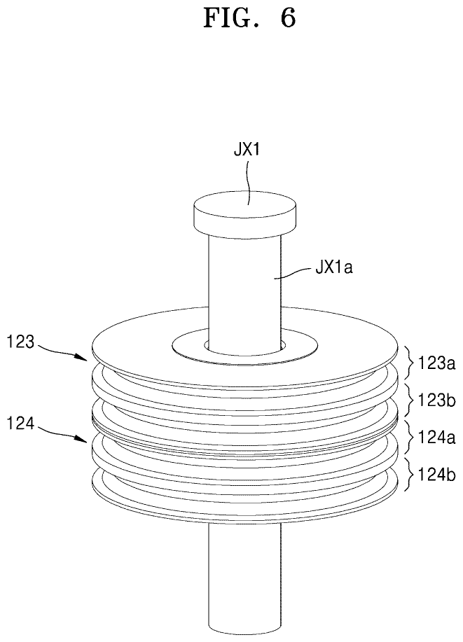

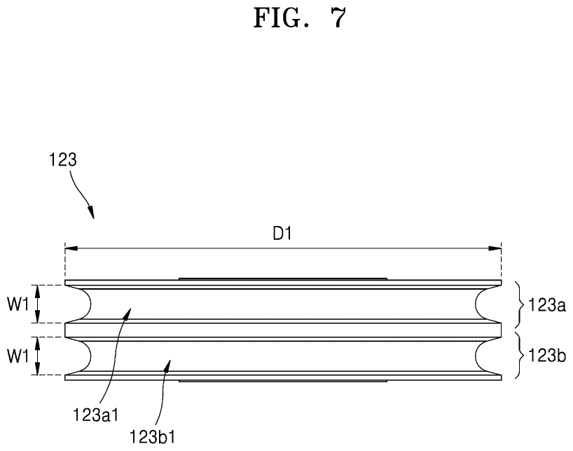

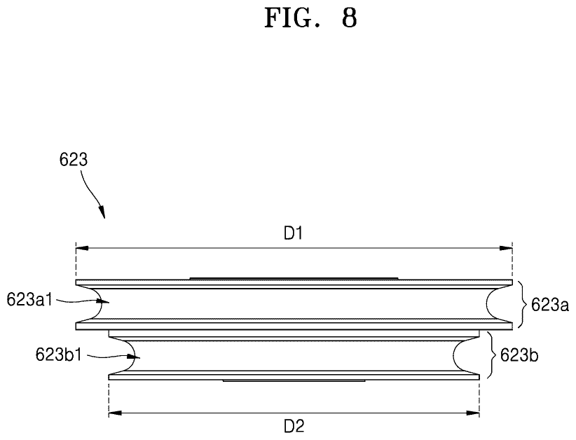

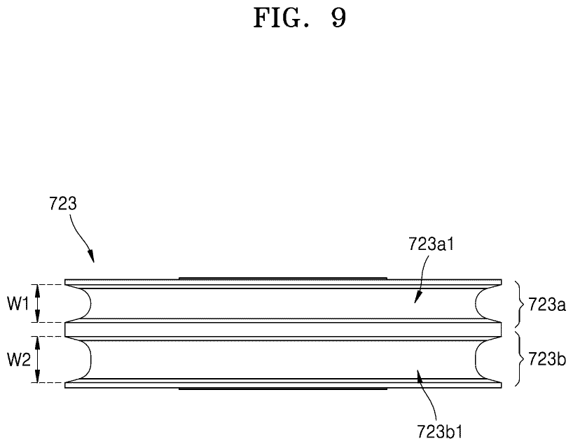

FIG. 5 is a perspective view for describing a rotational shaft and pulley structure of the end tool of FIG. 3, and FIG. 6 is a perspective view illustrating a double-groove pulley and the rotational shaft according to an embodiment of the present disclosure. FIG. 7 is a front view illustrating the double-groove pulley according to an embodiment of the present disclosure. FIG. 8 is a front view illustrating a double-groove pulley according to another embodiment of the present disclosure. FIG. 9 is a front view illustrating a double-groove pulley according to another embodiment of the present disclosure.

Referring to FIGS. 4 and 5, in the end tool according to an embodiment of the present disclosure, a plurality of rotational shafts may be disposed in the end tool hub 180 and the pitch hub 107. In an embodiment, in the end tool hub 180, the first rotational shaft JX1 may be positioned adjacent to the first jaw 101, and the second rotational shaft JX2, which is parallel to the first rotational shaft JX1, may be positioned adjacent to the first rotational shaft JX1. That is, the first rotational shaft JX1 and the second rotational shaft JX2 may be sequentially disposed from the proximal end 100p toward the distal end 100d.

Further, in the pitch hub 107, the third rotational shaft JX3 axially coupled to the end tool hub 180 may be disposed, and the fourth rotational shaft JX4 that is parallel to the third rotational shaft JX3 may be positioned adjacent to the third rotational shaft JX3.

In some embodiments, pulleys may be axially coupled to the first to fourth rotational shafts JX1 to JX4. In some embodiments, a first jaw pulley 111 and a second jaw pulley 112 may be axially coupled to the first rotational shaft JX1. This will be described in detail later.

Further, double-groove pulleys may be axially coupled to the first rotational shaft JX1. For example, a first double-groove pulley 123 adjacent to the first jaw pulley 111 and a second double-groove pulley 124 adjacent to the second jaw pulley 112 may be disposed. That is, the first jaw pulley 111, the first double-groove pulley 123, the second double-groove pulley 124, and the second jaw pulley 112 may be axially coupled to the same rotational shaft.

Referring further to FIGS. 6 and 7, the double-groove pulley according to an embodiment of the present disclosure may include a first pulley part 123a and a second pulley part 123b.

In some embodiments, the first pulley part 123a may include a first groove 123a1, and the second pulley part 123b may include a second groove 123b1. In some embodiments, the second groove 123b1 may be configured to be spaced apart from the first groove 123a1.

In some embodiments, the first pulley part 123a and the second pulley part 123b may be integrally formed.

From another perspective, the double-groove pulley may include the first groove 123a1 and the second groove 123b1 configured to be spaced apart from each other. In other words, the first groove 123a1 and the second groove 123b1 may be formed sequentially in a thickness direction of the pulley. The first groove 123a1 and the second groove 123b1 may each be a portion around which a wire is wound. In some embodiments, a first wire FW1 may be wound around the first groove 123a1, and a second wire RW1 may be wound around the second groove 123b1. Alternatively, the first wire FW1 may be wound around the second groove 123b1, and the second wire RW1 may be wound around the first groove 123a1. In other words, different wires may be at least partially in contact with the first groove 123a1 and the second groove 123b1, respectively. In some embodiments, a traveling path of each wire may vary depending on the double-groove pulley.

That is, the double-groove pulley is disposed such that at least a portion of the wire unit is in contact therewith, thereby guiding the traveling path of the wire unit. This will be described in detail later.

Referring to FIG. 7, in an embodiment, a width of the first groove 123a1 and a width of the second groove 123b1 of the double-groove pulley may be the same. In this case, a diameter of the first wire FW1 wound around the first groove 123a1 and a diameter of the second wire RW1 wound around the second groove 123b1 may be substantially the same. In some embodiments, a diameter D1 of each of the first pulley part 123a and the second pulley part 123b of the double-groove pulley may be the same.

In another embodiment, the diameter of the first pulley part 123a and the diameter of the second pulley part 123b of the double-groove pulley may be different from each other. Referring to FIG. 8, a diameter of a second pulley part 623b may be smaller than a diameter of a first pulley part 623a. In other words, the portion in which a second groove 623b1 is formed may have a smaller diameter than the portion in which a first groove 623a1 is formed. As such, the double-groove pulley may have a shape in which two pulleys of different diameters are combined.

In another embodiment, the width of the first groove 123a1 and the width of the second groove 123b1 of the double-groove pulley may be different from each other. Referring to FIG. 9, a width of a second groove 723b1 may be larger than a width of a first groove 723a1. In other words, a thickness of a second pulley part 723b may be greater than a thickness of a first pulley part 723a. In this case, a diameter of the second wire RW1 wound around the second groove 723a1 may be greater than a diameter of the first wire FW1 wound around the first groove 723a1. As such, the double-groove pulley may have a shape in which two pulleys, each having a groove of a different width, are combined.

However, the concept of the present disclosure is not necessarily limited thereto, and the number of grooves formed in one pulley, the width of the groove, and the diameter of the pulley part can be changed in various ways.

As described above, the double-groove pulley according to an embodiment of the present disclosure may be formed by combining one or more pulleys into a single component, thereby increasing the width of the single pulley.

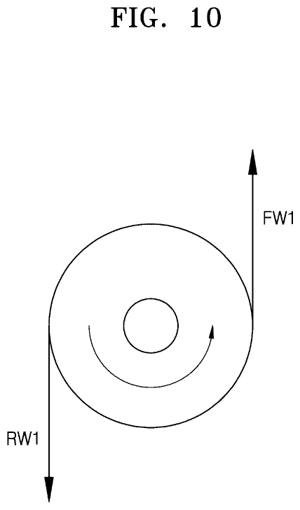

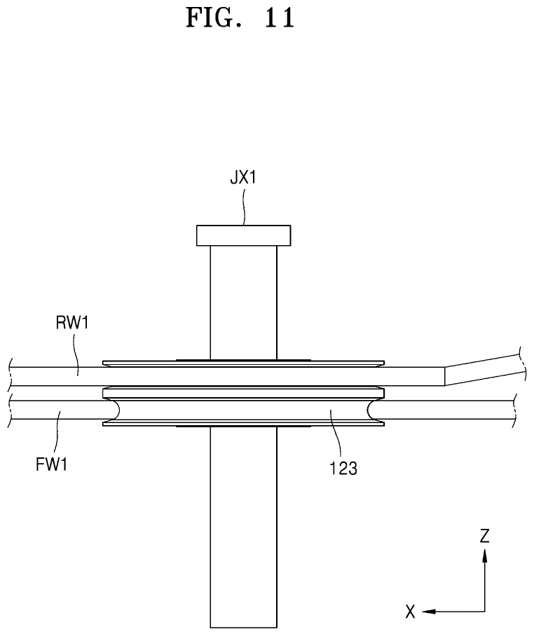

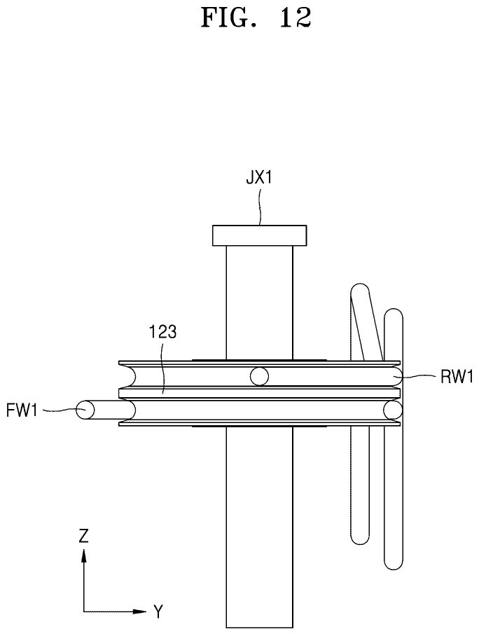

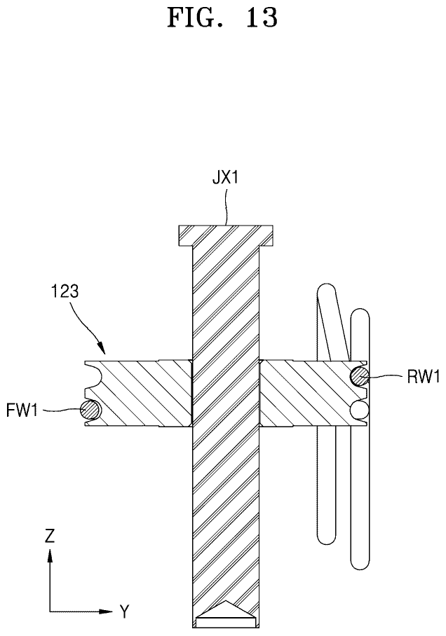

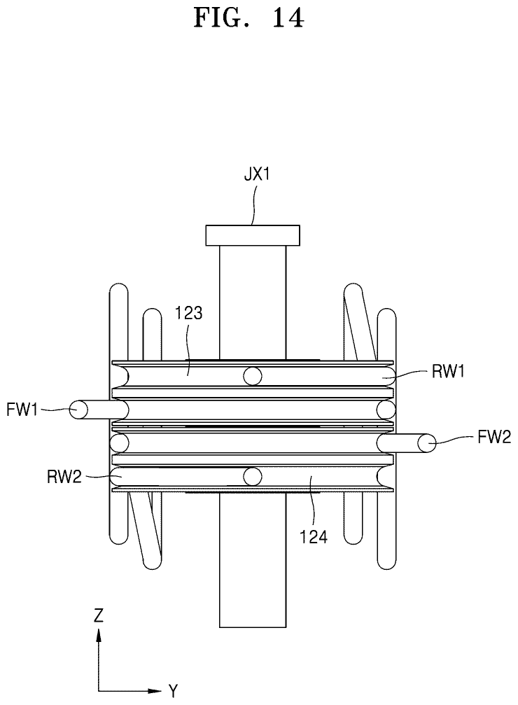

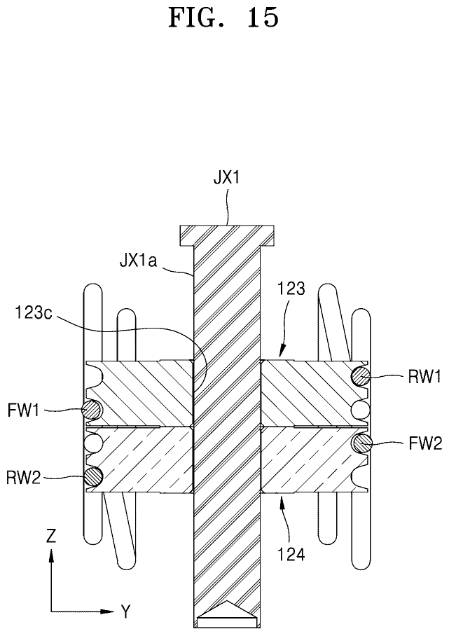

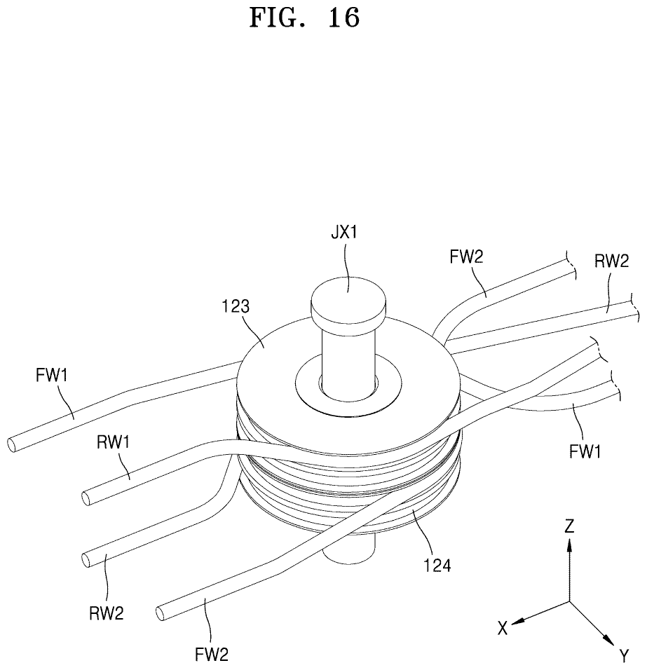

FIG. 10 is a view for describing a relationship between the double-groove pulley and the wires according to an embodiment of the present disclosure. FIG. 11 is a side view schematically illustrating the first double-groove pulley 123 and the wires of the end tool of FIG. 3, FIG. 12 is a front view of the first double-groove pulley 123 of FIG. 11 as viewed from one direction, and FIG. 13 is a cross-sectional view illustrating a cross section of the first double-groove pulley 123 of FIG. 11. FIG. 14 is a front view schematically illustrating the first and second double-groove pulleys 123 and 124 and the wires of the end tool of FIG. 3, and FIG. 15 is a cross-sectional view illustrating cross sections of the first double-groove pulley 123 and the second double-groove pulley 124 of FIG. 14. FIG. 16 is a perspective view schematically illustrating the first and second double-groove pulleys 123 and 124 and the wires of the end tool of FIG. 3.

Referring to FIGS. 10 to 13, in an embodiment, the first wire FW1 may be positioned on one side of the rotational shaft of the first double-groove pulley 123, while the second wire RW1 may be positioned on another side of the rotational shaft of the first double-groove pulley 123.

In other words, a pair of wires wound around the double-groove pulley may be positioned in opposite directions with respect to the rotational shaft.

In some embodiments, when the double-groove pulley rotates in one direction, the first and second wires FW1 and RW1 wound around the double-groove pulley may move in different directions.

In some embodiments, as illustrated in FIG. 10, when the double-groove pulley rotates in a counterclockwise direction, the first wire FW1 wound around a right side of the double-groove pulley may move upward based on the drawing, and the second wire RW1 wound around a left side of the double-groove pulley may move downward. In other words, when the first wire FW1 and the second wire RW1 move, both wires may cause the double-groove pulley to rotate in the same direction.

As shown in FIGS. 11 to 13, the first wire FW1 may be positioned in a groove formed at a lower side of the first double-groove pulley 123, and the second wire RW1 may be positioned in a groove formed at an upper side of the first double-groove pulley 123.

Referring to FIGS. 14 and 15, in an embodiment, the first double-groove pulley 123 and the second double-groove pulley 124 may be axially coupled to one rotational shaft. In some embodiments, based on the rotational shaft, a third wire FW2 may be positioned on one side of the rotational shaft, and a fourth wire RW2 may be positioned on another side of the rotational shaft.

In some embodiments, when the second double-groove pulley 124 rotates in one direction, the third wire FW2 and the fourth wire RW2 wound around the second double-groove pulley 124 may move in different directions.

The third wire FW2 may be positioned in a groove formed at an upper side of the second double-groove pulley 124, and the fourth wire RW2 may be positioned in a groove formed at a lower side of the second double-groove pulley 124.

The first wire FW1 and the fourth wire RW2 may be positioned on one side with respect to the rotational shaft, and the second wire RW1 and the third wire FW2 may be positioned on another side with respect to the rotational shaft.

In other words, the first wire FW1 and the third wire FW2 may be positioned on different sides with respect to the rotational shaft. In some embodiments, the second wire RW1 and the fourth wire RW2 may be positioned on different sides with respect to the rotational shaft.

In another embodiment, the first wire FW1 and the second wire RW1 may be positioned on the same side with respect to the rotational shaft of the double-groove pulley.

In other words, a pair of wires wound around the double-groove pulley may be positioned in the same direction with respect to the rotational shaft.

In this case, when the double-groove pulley rotates in one direction, the first wire FW1 and the second wire RW1 wound around the double-groove pulley may move in the same direction.

Hereinafter, the application of the above-described double-groove pulley to the end tool 100 according to an embodiment of the present disclosure will be described.

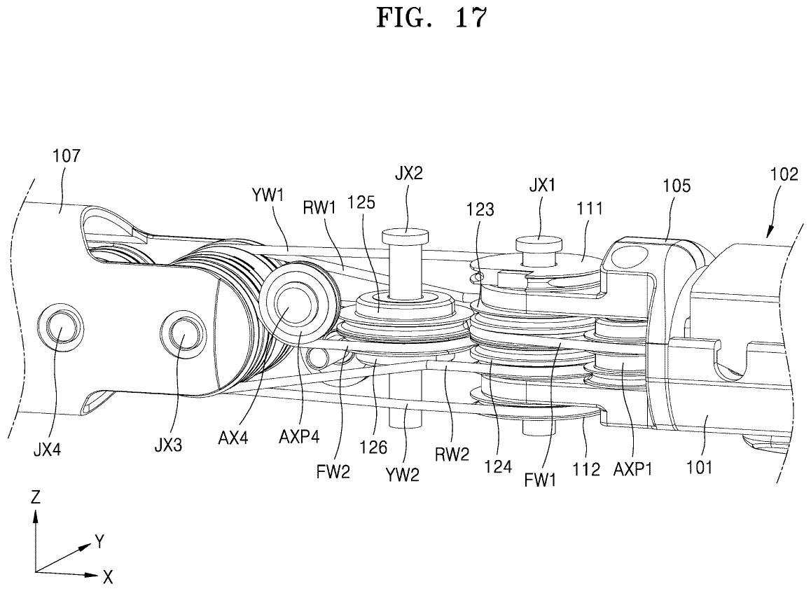

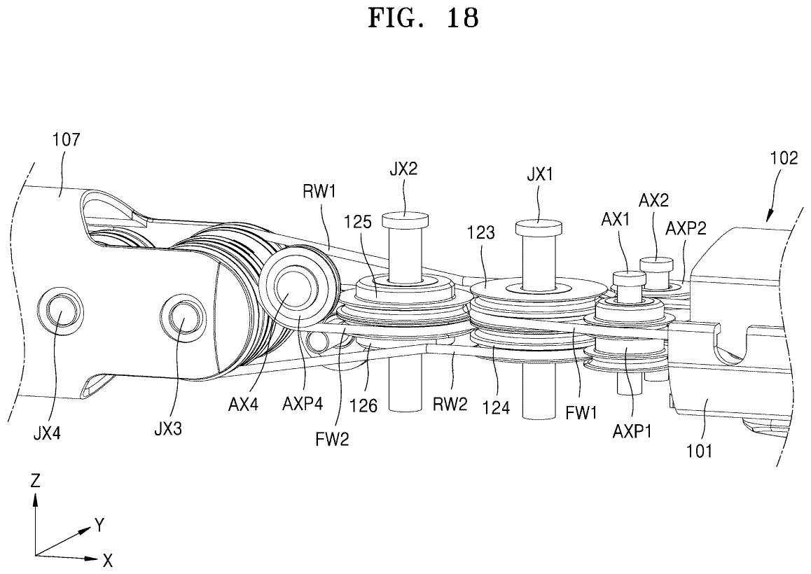

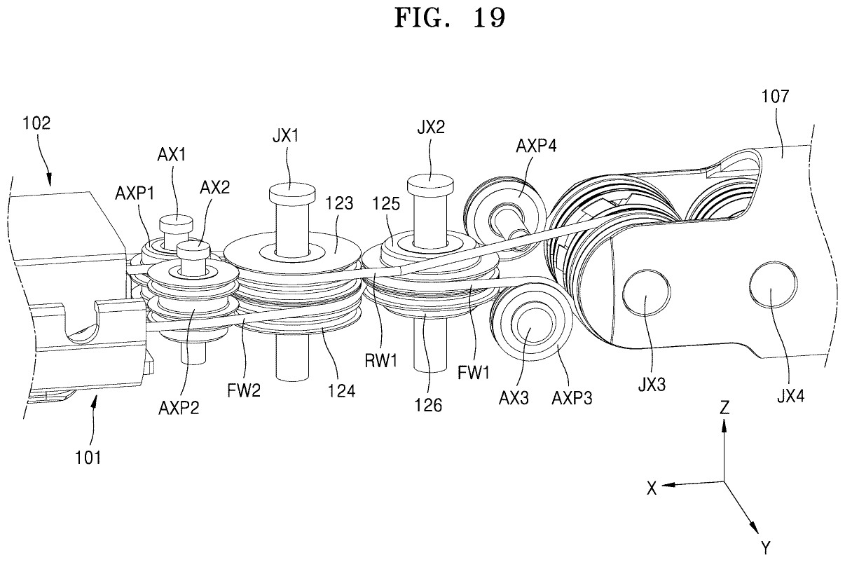

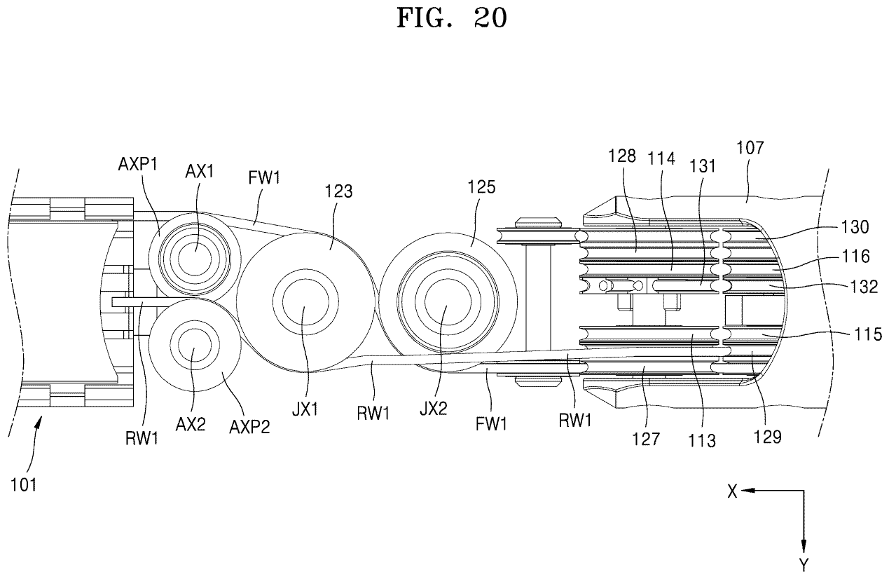

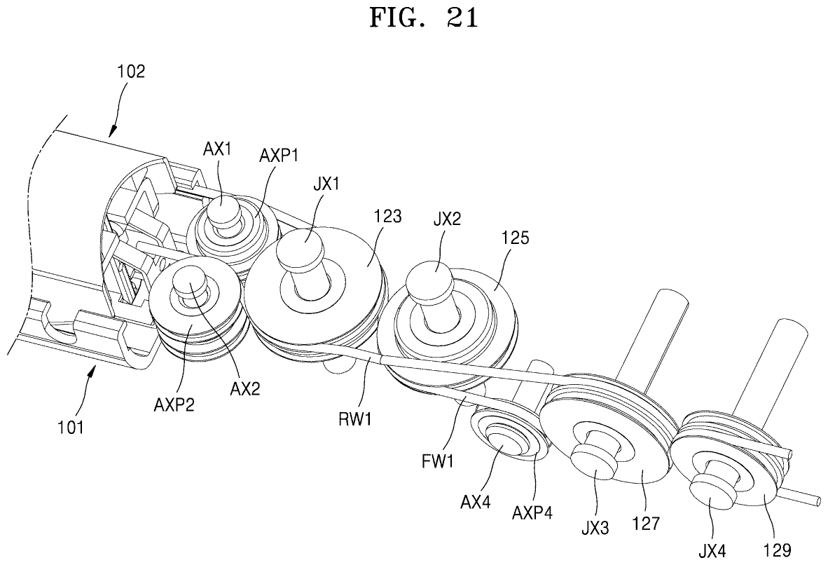

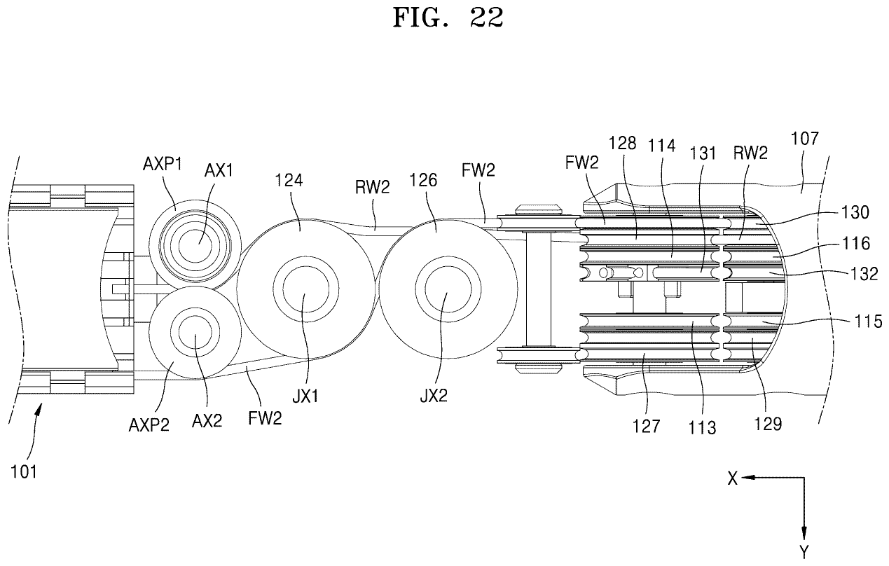

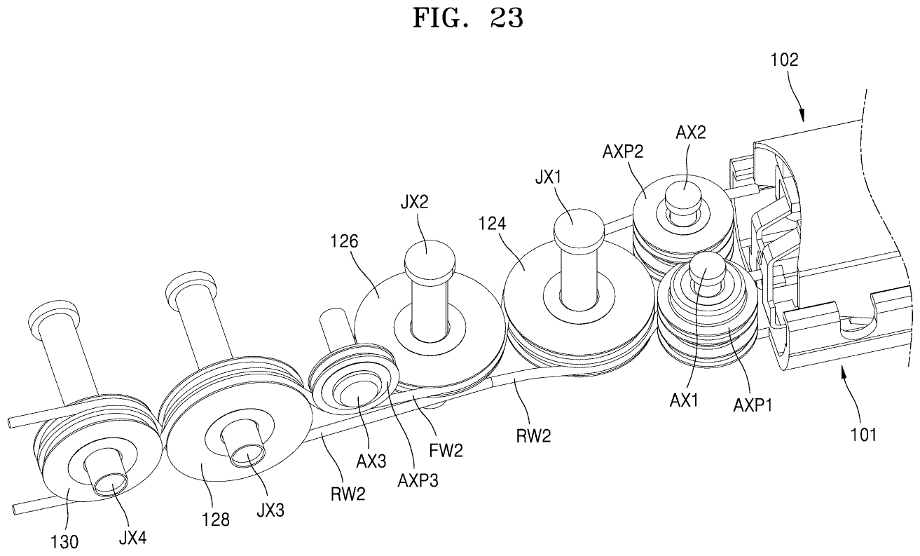

FIG. 17 is a perspective view illustrating a state in which the end tool hub 180 of the end tool of FIG. 3 is removed, and FIGS. 18 and 19 are perspective views illustrating a state in which the jaw pulley is removed from the end tool of FIG. 17. FIG. 20 is a plan view for describing paths of the wires wound around the first double-groove pulley 123 in the end tool of FIG. 3, and FIG. 21 is a perspective view of the end tool of FIG. 20 viewed from another direction. FIG. 22 is a plan view for describing paths of the wires wound around the second double-groove pulley 124 in the end tool of FIG. 3, and FIG. 23 is a perspective view of the end tool of FIG. 22 viewed from another direction.

Referring to FIGS. 4, 5, and 17 to 19, the first rotational shaft JX1 may connect the end tool hub 180 to the first jaw 101. In some embodiments, the first jaw 101 may include a jaw connection part 105, and the jaw connection part 105 may include a first shaft coupling part 105a and a second shaft coupling part 105b, which extend toward the end tool hub 180.

In some embodiments, the first rotational shaft JX1 may pass through the jaw pulley coupling parts 181 and 182 of the end tool hub 180, the first shaft coupling part 105a, and the second shaft coupling part 105b to axially couple the end tool hub 180 to the jaw connection part 105. In some embodiments, the first rotational shaft JX1 may function as a yaw rotational shaft. In other words, the end tool hub 180 and the jaw connection part 105 may relatively rotate around the first rotational shaft JX1.

The first jaw pulley 111 may be positioned between the jaw pulley coupling part 181 at the upper side and the first shaft coupling part 105a, and the second jaw pulley 112 may be positioned between the jaw pulley coupling part 182 at the lower side and the second shaft coupling part 105b.

In some embodiments, as a first yaw wire YW1 is pulled toward the proximal end 100p, the first jaw pulley 111 is rotated, and a rotational force may be transmitted to the first shaft coupling part 105a coupled to the first jaw pulley 111. Likewise, when a second yaw wire YW2 is pulled toward the proximal end 100p, the second jaw pulley 112 is rotated, and a rotational force may be transmitted to the second shaft coupling part 105b coupled to the second jaw pulley 112.

In some embodiments, the first double-groove pulley 123 and the second double-groove pulley 124 may be positioned between the first shaft coupling part 105a and the second shaft coupling part 105b. That is, the first rotational shaft JX1 may pass through the first double-groove pulley 123 and the second double-groove pulley 124 to be axially coupled to the first shaft coupling part 105a and the second shaft coupling part 105b.

In other words, the first and second double-groove pulleys 123 and 124 may be axially coupled to the same rotational shaft as the first jaw pulley 111 and the second jaw pulley 112. In other words, the first double-groove pulley 123 and the second double-groove pulley 124 may be axially coupled to the yaw rotational shaft.

In some embodiments, single-groove pulleys may be axially coupled to the second rotational shaft JX2. In some embodiments, a plurality of pulleys, in each of which one groove is formed, may be disposed. In some embodiments, a first auxiliary pulley 125 and a second auxiliary pulley 126 may be axially coupled to the second rotational shaft JX2.

Further, the single-groove pulleys may also be disposed on the end tool hub 180 and the pitch hub 107. In some embodiments, a pulley 113, a pulley 114, a pulley 115, a pulley 116, a pulley 131, and the pulley 132 may be single-groove pulleys.

In some embodiments, the pulley 113, the pulley 114, and the pulley 131 may each be axially coupled to the third rotational shaft JX3, and the pulley 115, the pulley 116, and the pulley 132 may each be axially coupled to the fourth rotational shaft JX4.

The first yaw wire YW1 may sequentially come into contact with the first jaw pulley 111, the pulley 113, and the pulley 115 while moving from the distal end 100d toward the proximal end 100p of the end tool.

The second yaw wire YW2 may sequentially come into contact with the second jaw pulley 112, pulley 114, and pulley 116 while moving from the distal end 100d toward the proximal end 100p of the end tool.

In some embodiments, the pulley 131 may function as a pitch pulley, and pulley 132 may function as a pitch auxiliary pulley.

In some embodiments, the double-groove pulleys may also be disposed on the pitch hub 107. For example, a third double-groove pulley 127 and a fourth double-groove pulley 128 may be axially coupled to the third rotational shaft JX3, and a fifth double-groove pulley 129 and a sixth double-groove pulley 130 may be axially coupled to the fourth rotational shaft JX4.

In some embodiments, based on an imaginary plane passing through the first rotational shaft JX1 and the second rotational shaft JX2, the third double-groove pulley 127 may be positioned on one side and the fourth double-groove pulley 128 may be positioned on another side. In other words, the third double-groove pulley 127 may be positioned on one side of the pitch hub 107 and the fourth double-groove pulley 128 may be positioned on another side of the pitch hub 107.

In some embodiments, the third rotational shaft JX3 may pass through the pitch pulley coupling part 185 of the end tool hub 180 to axially couple and secure the end tool hub 180 and the pitch hub 107. That is, the third rotational shaft JX3 may function as a pitch rotational shaft. In other words, the third double-groove pulley 127 and the fourth double-groove pulley 128 may be axially coupled to the pitch rotational shaft.

In some embodiments, the fifth double-groove pulley 129 may be positioned adjacent to the third double-groove pulley 127, and the sixth double-groove pulley 130 may be positioned adjacent to the fourth double-groove pulley 128.

In some embodiments, the fifth double-groove pulley 129 and the sixth double-groove pulley 130 may function as pitch auxiliary pulleys.

In some embodiments, as described above, the wire unit may include the first wire FW1, the second wire RW1, the third wire FW2, and the fourth wire RW2.

The first wire FW1 and the second wire RW1 may be wound around the first double-groove pulley 123, and the third wire FW2 and the fourth wire RW2 may be wound around the second double-groove pulley 124.

In some embodiments, the first wire FW1 and the second wire RW1 may may constitute a pair of wires, and the third wire FW2 and the fourth wire RW2 may may constitute a pair of wires.

In some embodiments, a pair of wires may be wound around the double-groove pulley, and the pair of wires may manipulate one degree of freedom of the jaw 103.

In some embodiments, the first jaw 101 may be configured to accommodate at least one region of an operation member (not shown) that is movable in at least one direction. In some embodiments, the first wire FW1 and the second wire RW1 may be connected to the operation member so that the operation member can move in at least one direction. The first wire FW1 and the second wire RW1 may control the degrees of freedom of the operation member.

For example, the first wire FW1 may be a firing wire that moves the operation member toward the distal end 100d of the end tool, and the second wire RW1 may be a retraction wire that moves the operation member toward the proximal end 100p of the end tool.

That is, the first wire FW1 and the second wire RW1 may be wires that control the degrees of freedom related to the forward or backward movement of the operation member.

In some embodiments, the first wire FW1 and the second wire RW1 may move in different directions and may move the operation member forward or backward. Accordingly, the first wire FW1 and the second wire RW1 may move in opposite directions at a 1:1 ratio.

As the first wire FW1 and the second wire RW1 move in opposite directions at a 1:1 ratio as described above, the first wire FW1 and the second wire RW1 may be positioned on opposite sides of the rotational shaft when wound around the first double-groove pulley 123.

By positioning the first wire FW1 and the second wire RW1 as described above, the first double-groove pulley 123 may rotate in one direction when the first wire FW1 and the second wire RW1 move.

In other words, a direction in which the first double-groove pulley 123 is rotated by the first wire FW1 and a direction in which the first double-groove pulley 123 is rotated by the second wire RW1 may be the same.

Similarly, the third wire FW2 and the fourth wire RW2 may be connected to the operation member so that the operation member can move in at least one direction. The third wire FW2 and the fourth wire RW2 may control the degrees of freedom of the operation member.

For example, the third wire FW2 may be a firing wire that moves the operation member toward the distal end 100d of the end tool, and the fourth wire RW2 may be a retraction wire that moves the operation member toward the proximal end 100p of the end tool.

That is, the third wire FW2 and the fourth wire RW2 may be wires that control the degrees of freedom related to the forward or backward movement of the operation member.

In some embodiments, the third wire FW2 and the fourth wire RW2 move in different directions and may move the operation member forward or backward. Accordingly, the third wire FW2 and the fourth wire RW2 may move in opposite directions at a 1:1 ratio.

As the third wire FW2 and the fourth wire RW2 move in opposite directions at a 1:1 ratio as described above, when the third wire FW2 and the fourth wire RW2 are wound around the second double-groove pulley 124, the third wire FW2 and the fourth wire RW2 may be positioned on opposite sides of the rotational shaft.

By positioning the third wire FW2 and the fourth wire RW2 as described above, when the third wire FW2 and the fourth wire RW2 move, the second double-groove pulley 124 may rotate in one direction.

In other words, a direction in which the second double-groove pulley 124 is rotated by the third wire FW2 and a direction in which the second double-groove pulley 124 is rotated by the fourth wire RW2 may be the same.

In some embodiments, the first wire FW1 and the second wire RW1 may sequentially come into contact with the first double-groove pulley 123, the third double-groove pulley 127, and the fifth double-groove pulley 129 while moving from the distal end 100d of the end tool toward the proximal end 100p.

In some embodiments, the third wire FW2 and the fourth wire RW2 may sequentially come into contact with the second double-groove pulley 124, the fourth double-groove pulley 128, and the sixth double-groove pulley 130.

In some embodiments, with respect to the third rotational shaft JX3, which is a rotational shaft of the third double-groove pulley 127, the first wire FW1 may be positioned on one side, and the second wire RW1 may be positioned on another side. In some embodiments, with respect to the fourth rotational shaft JX4, the first wire FW1 may be positioned on one side, and the second wire RW1 may be positioned on another side.

With respect to the third rotational shaft JX3, which is a rotational shaft of the fourth double-groove pulley 128, the third wire FW2 may be positioned on one side, and the fourth wire RW2 may be positioned on another side. In some embodiments, with respect to the fourth rotational shaft JX4, the third wire FW2 may be positioned on one side, and the fourth wire RW2 may be positioned on another side.

In some embodiments, referring to FIGS. 20 and 21, the first wire FW1, which is a first firing wire, may extend in a direction toward the proximal end 100p from the first jaw 101, come into contact with one region of the first double-groove pulley 123, and wind around the first rotational shaft JX1 from the right side before exiting to the left side and coming into contact with a first auxiliary pulley 125. In some embodiments, the first wire FW1 may come into contact with one region of the third double-groove pulley 127 after passing from the first auxiliary pulley 125, wind around the third double-groove pulley 127 from the lower side, exit from the upper side of the third double-groove pulley 127, then come into contact with one region of the fifth double-groove pulley 129, wind around the fifth double-groove pulley 129 from the upper side, and extend in a direction toward the proximal end 100p.

The second wire RW1, which is a first retraction wire, may extend from the first jaw 101 toward the proximal end 100p, come into contact with one region of the first double-groove pulley 123, wind around the first rotational shaft JX1 from the left side before exiting to the left side, then come into contact with one region of the third double-groove pulley 127, wind around the third double-groove pulley 127 from the upper side before exiting to the lower side, then come into contact with one region of the fifth double-groove pulley 129, wind around the fifth double-groove pulley 129 from the lower side, and extend in a direction toward the proximal end 100p.

Referring to FIGS. 22 and 23, the third wire FW2, which is a second firing wire, may extend in a direction toward the proximal end 100p from the first jaw 101, come into contact with one region of the second double-groove pulley 124, wind around the first rotational shaft JX1 from the left side before exiting to the right side, and come into contact with the second auxiliary pulley 126. In some embodiments, the third wire FW2 may come into contact with one region of the fourth double-groove pulley 128 after passing from the second auxiliary pulley 126, wind around the fourth double-groove pulley 128 from the lower side, exit from the upper side of the fourth double-groove pulley 128, then come into contact with one region of the sixth double-groove pulley 130, wind around the sixth double-groove pulley 130 from the upper side, and extend in a direction toward the proximal end 100p.

The fourth wire RW2, which is a second retraction wire, may extend from the first jaw 101 toward the proximal end 100p, come into contact with one region of the second double-groove pulley 124, wind around the first rotational shaft JX1 from the right side before exiting to the right side, then come into contact with one region of the fourth double-groove pulley 128, wind around the fourth double-groove pulley 128 from the lower side before exiting to the upper side, then come into contact with one region of the sixth double-groove pulley 130, wind around the sixth double-groove pulley 130 from the upper side, and extend in a direction toward the proximal end 100p.

As described above, in the end tool according to an embodiment of the present disclosure, by positioning the double-groove pulley at the joint part, space utilization can be increased.

In the related art, each pulley was configured to have only one wire wound therearound. In general, while the number of wires required for product operation is predetermined, the space available for accommodating pulleys is limited, thereby restricting the width of the pulleys.

In some embodiments, bearings are used to reduce pulley friction, and the performance of the bearings is greatly influenced by the size of bearing balls. However, the size of the usable bearing balls is determined by a space between the outer and inner diameters of the bearing, as well as a width of the bearing. Even when the space between the outer and inner diameters of the bearing is sufficient, the diameter of the usable bearing balls may still be limited by the width of the bearing when the width is too small. Accordingly, in this case, sufficiently large bearing balls cannot be used, leading to a decrease in a load capacity of the bearing and potentially causing excessive friction and noise.

In some embodiments, the end tool according to an embodiment of the present disclosure employs the double-groove pulley, which increases the usable width of the bearing, thereby enabling the use of bearing balls with a larger diameter. Accordingly, the durability of the bearing is improved, and the maximum load capacity may increase.

Further, the end tool according to an embodiment of the present disclosure employs the double-groove pulley, which can increase the width of the pulley, thereby enlarging a contact area between the pulley and the rotational shaft. Accordingly, as a surface pressure acting between an inner surface of the pulley and the rotational shaft decreases, the likelihood of damage to components, such as surface scratches, is reduced, and the durability and performance of surface coating applied to the pulley or the rotational shaft may be improved. That is, the end tool according to an embodiment of the present disclosure employs the double-groove pulley, which can minimize a frictional force resisting pulley rotation and can also be effective in reducing the number of components.

In some embodiments, through a structure in which a pair of wires are positioned on opposite sides of the rotational shaft, a portion of the force components directed toward the center from the pulley's diameter, which are exerted by each wire on the rotational shaft, may cancel each other out, and as a result, a surface pressure between the pulley and the rotational shaft is reduced, thereby decreasing friction.

In some embodiments, the double-groove pulley according to an embodiment of the present disclosure may, of course, be applied not only to the end tool but also to other areas in which a wire and pulley structure is required. For example, the double-groove pulley structure may, of course, also be applied to the manipulation part or the power transmission part for driving the end tool.

Hereinafter, an end tool according to another embodiment of the present disclosure will be described. Descriptions of the cartridge, the operation member, and the like of the end tool provided below may also be applied to the above-described embodiment.

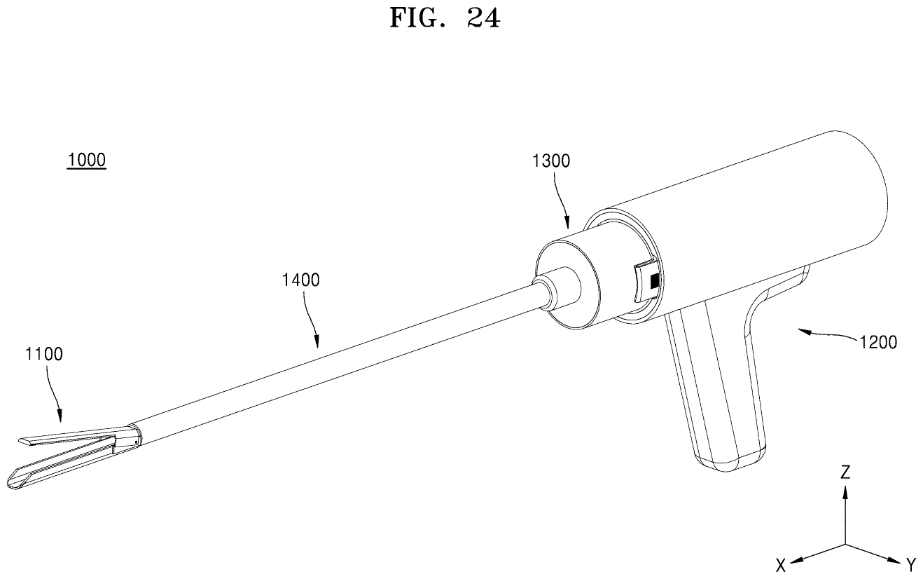

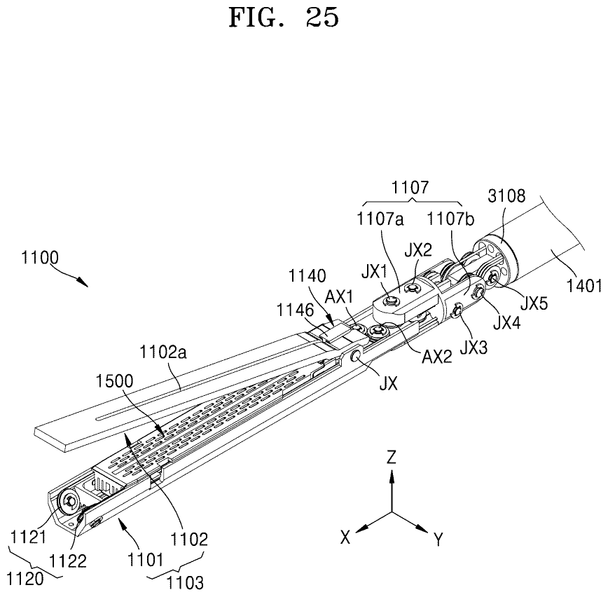

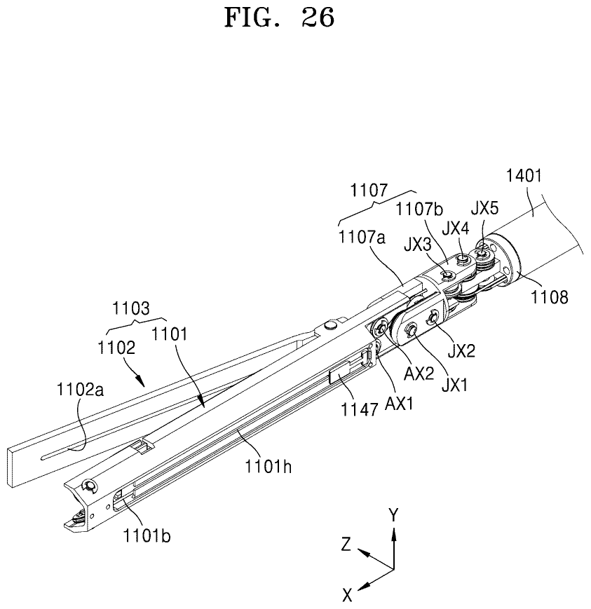

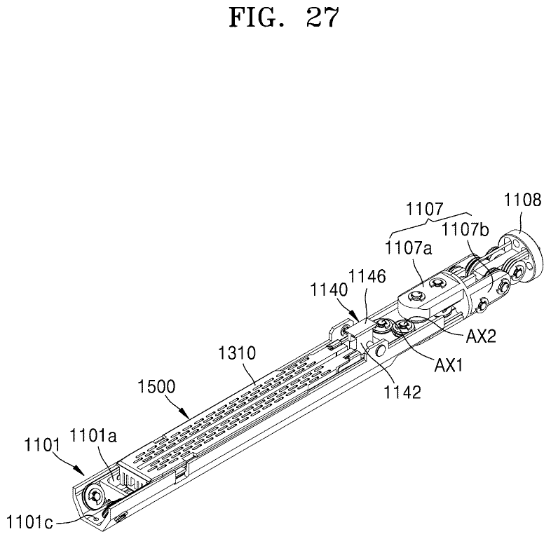

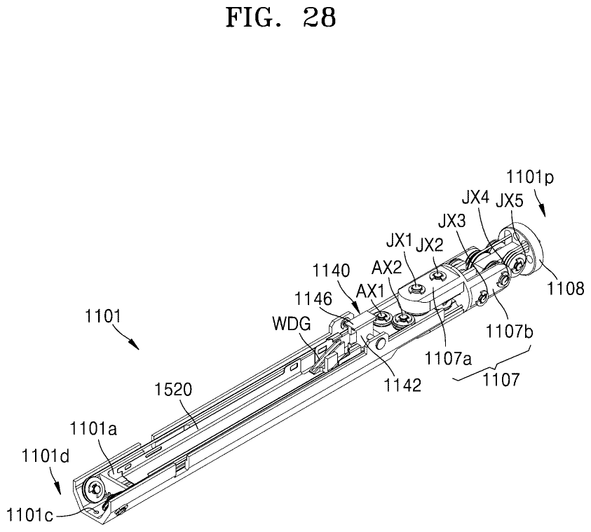

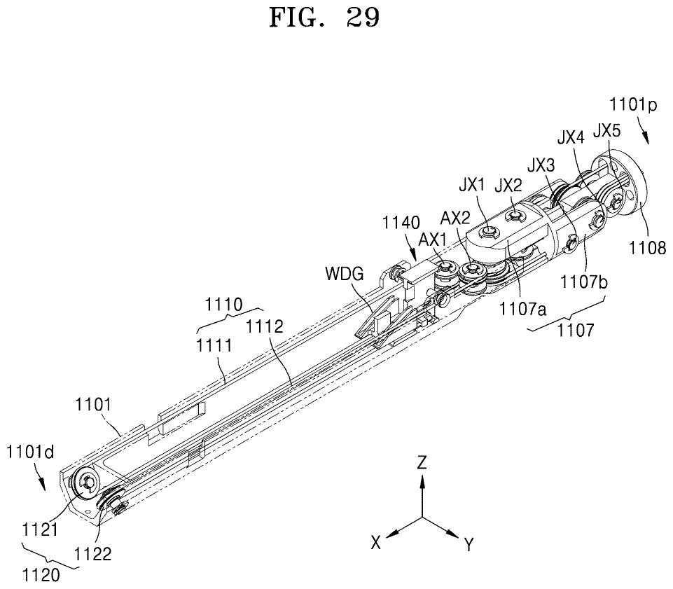

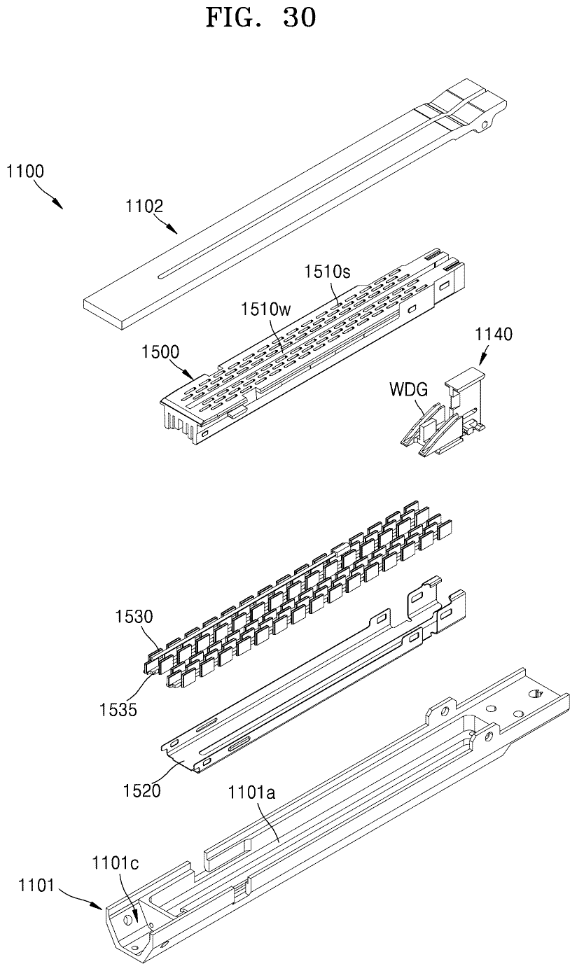

FIG. 24 is a perspective view schematically illustrating a surgical instrument to which the end tool according to another embodiment of the present disclosure is applied, FIG. 25 is a schematic perspective view for describing the end tool of FIG. 24, and FIG. 26 is a perspective view of the end tool of FIG. 25 viewed from another direction. FIG. 27 is a schematic perspective view of the end tool of FIG. 25 with a second jaw removed, FIG. 28 is a schematic perspective view of the end tool of FIG. 27 with a cartridge removed, and FIG. 29 is a transparent perspective view of FIG. 28. FIG. 30 is a perspective view illustrating a first jaw and the cartridge of the surgical instrument of FIG. 24.

An end tool 1100 may include a jaw 1103, a plurality of fixed pulleys 1120, and a plurality of firing wires 1110. The plurality of fixed pulleys 1120 may include two or more pulleys, for example, a first fixed pulley 1121 and a second fixed pulley 1122. The plurality of firing wires 1110 include two or more wires, and may include, for example, a first firing wire 1111 and a second firing wire 1112.

The jaw 1103 may perform various functions, for example, a grip motion, and may include a pair of jaws, e.g., a first jaw 1101 and a second jaw 1102 as a specific example. In some embodiments, each of the first jaw 1101 and the second jaw 1102, or a component encompassing the first jaw 1101 and the second jaw 1102 may be referred to as the jaw 1103.

The first jaw 1101 and the second jaw 1102 may be disposed to face each other, may move closer to or move away from each other, and may be configured to rotationally move around, for example, one shaft JX.

A cartridge 1500 may be disposed to be accommodated in the first jaw 1101, and a plurality of staples are positioned inside the cartridge 1500. When an operation member 1140 receives a force through the plurality of firing wires 1110 while the first jaw 1101 and the second jaw 1102 are close to each other, such as when the first jaw 1101 and the second jaw 1102 are closed with body tissue interposed therebetween, the operation member 1140 may push and raise the staples while moving toward a distal end 1101d of the first jaw 1101, so that stapling may be performed. At this point, one or more clamps 1146 and 1147 of the operation member 1140 may protrude to the outside of the first jaw 1101 and the second jaw 1102, allowing the operation member 1140 to move forward while applying pressure to outer surfaces of the first jaw 1101 and the second jaw 1102, which facilitates the smooth progression of a stapling process. In an optional embodiment, the cartridge 1500 may include a case 1520 corresponding to the bottom, and the case 1520 is disposed in the first jaw 1101.

In some embodiments, the operation member 1140 may be used together with a wedge WDG. For example, the wedge WDG may be prepared separately from the operation member 1140 and then positioned adjacent to the operation member 1140 in the first jaw 1101. In some embodiments, as another example, the operation member 1140 and the wedge WDG may be integrally formed. The wedge WDG may be disposed on at least one side of a body 1142 and may be formed to have a predetermined inclined surface. That is, the wedge WDG may be formed to be inclined by a certain degree with respect to an extension direction of the end tool 1100. In other words, the wedge WDG may be formed to have a greater height at a proximal end 1101p side of the first jaw 1101 than the distal end 1101d side of the first jaw 1101.

The wedge WDG may be configured to be sequentially in contact with withdrawal members 1535 or a plurality of staples 1530 positioned in the cartridge 1500, and may serve to sequentially push and raise the staples 1530.

The plurality of fixed pulleys 1120 may be disposed in the first jaw 1101 to be closer to the front of the cartridge 1500, i.e., to the distal end 1101d of the first jaw 1101, than the cartridge 1500. For example, the plurality of fixed pulleys 1120 may be disposed in a front space 1101c of the first jaw 1101, and details thereof will be described below.

In some embodiments, the end tool 1100 for the surgical instrument of the present embodiment may include one or more members, such as joint members, that connect the jaw 1103 to a connection part 1400. Further, in an optional embodiment, the end tool 1100 may include an end tool hub 1108 and a pitch hub 1107.

The end tool hub 1108 may be disposed to connect the end tool 1100 to a straight part 1401 of the connection part 1400.

As an example, the end tool hub 1108 may have a pulley shaft JX4 corresponding thereto, and the pulley shaft JX4 may be a pitch rotational shaft. As a specific example, the end tool 1100 may perform a vertical rotational motion around the pulley shaft JX4 based on the drawing. In some embodiments, one or more pulleys may be positioned to be adjacent to the pulley shaft JX4.

The end tool hub 1108 may be in the form of a bar extending from the center of a surface thereof that corresponds to the connection part 1400, i.e., a bar extending from the center of a disk-shaped main region. The pulley shaft JX4 and a pulley shaft JX5 different from the pulley shaft JX4 may further correspond to a region of the bar.

The pitch hub 1107 is connected to the end tool hub 1108 and the jaw 1103. The pitch hub 1107 may be axially coupled to the end tool hub 1108 with respect to one pulley shaft, i.e., the pulley shaft JX4. The pitch hub 1107 may rotationally move around one pulley shaft, i.e., the pulley shaft JX4 while connected to the end tool hub 1108. That is, the end tool 1100 may perform a pitch motion as the pitch hub 1107 rotates around one pulley shaft, i.e., the pulley shaft JX4 with respect to the end tool hub 1108.

Further, the jaw 1103 of the end tool 1100 may be axially coupled to the pitch hub 1107 with respect to one pulley shaft, i.e., a pulley shaft JX1. The jaw 1103 may rotate around one pulley shaft, i.e., the pulley shaft JX1 while connected to the pitch hub 1107. That is, the jaw 1103 of the end tool 1100 may rotate around one pulley shaft, i.e., the pulley shaft JX1 with respect to the pitch hub 1107, thereby performing a yaw motion.

As a result, the yaw motion of the end tool 1100 includes a rotational motion of the jaw 1103 around one pulley shaft, i.e., the pulley shaft JX1 with respect to the pitch hub 1107, and the pitch motion of the end tool 1100 includes a rotational motion of the jaw 1103 coupled to the pitch hub 1107, which occurs as the pitch hub 1107 rotates around one pulley shaft, i.e., the pulley shaft JX4 together with the end tool hub 1108.

The pitch hub 1107 may include a first hub 1107a and a second hub 1107b.

The first hub 1107a of the pitch hub 1107 may be connected to the jaw 1103. As an example, the first hub 1107a may be elongated to connect to one region of the first jaw 1101, and specifically, may have two bars that are formed side by side to face each other and coupled to each other by placing one region of the first jaw 1101 therebetween.

The second hub 1107b of the pitch hub 1107 may be connected to the end tool hub 1108, for example, may have two bars that are formed side by side to face each other, and may be coupled to each other by placing one region of the end tool hub 1108 therebetween.

As described above, the pulley shaft JX5 different from one pulley shaft, i.e., the pulley shaft JX4 may be disposed in the end tool hub 1108 to be spaced apart from the pulley shaft JX4 and closer to the connection part 1400 than the pulley shaft JX4. The pulley shaft JX4 and the pulley shaft JX5 may have axes in directions parallel to each other.

A pulley shaft JX2, which is different from the pulley shaft JX1, is disposed in the pitch hub 1107 in a direction adjacent to and parallel to the pulley shaft JX1. In some embodiments, a pulley shaft JX3 and the pulley shaft JX4 may be formed in a direction different from (for example, intersecting or orthogonal to) the direction in which the pulley shaft JX1 and the pulley shaft JX2 are disposed, and may be sequentially positioned in a direction toward (or away from the operation member) the connection part 1400.

The pulley shaft JX4 may be a pitch motion shaft of the end tool 1100, and the pulley shaft JX1 may be a yaw motion shaft.

The pulley shaft JX3 and the pulley shaft JX5 may be pitch auxiliary pulley shafts, and the pulley shaft JX2 may be a yaw auxiliary pulley shaft. One or more driving wires, such as a wire configured to transmit a driving force for a pitch motion or a yaw motion may have at least one region in contact with or wound around the pulley shafts JX1, JX2, JX3, JX4, and JX5.

The pulley shafts JX2, JX3, and JX5 adjacent to the pulley shaft JX4, which is a pitch motion shaft, and the pulley shaft JX1, which is a yaw motion shaft, may control paths along which the driving wires are wound around the pulley shaft JX4 and the pulley shaft JX1 to secure the efficiency of the arrangement of the driving wires and stabilize the paths for transmitting forces through the driving wires.

In some embodiments, at least one region of the firing wire 1110 may be in contact with or wound around the pulley shafts JX1, JX2, JX3, JX4, and JX5.

A more detailed description of the arrangement of the pulley shafts JX1, JX2, JX3, JX4, and JX5 will be provided below.

One or more switching pulley shafts AX1 and AX2 may be disposed in the end tool 1100, and one or more pulleys corresponding to the switching pulley shafts AX1 and AX2 may be disposed.

For example, the switching pulley shafts AX1 and AX2 may be disposed in the jaw 1103, specifically, at the proximal end 1101p side of the first jaw 1101, and may be disposed closer to the distal end 1101d of the first jaw 1101 than at least the above-described pulley shafts JX1, JX2, JX3, JX4, and JX5.

The switching pulley shafts AX1 and AX2 may be shafts formed parallel to each other, and may be disposed to have different backward and forward positions with respect to each other such that the switching pulley shaft AX1 and the switching pulley shaft AX2 are sequentially disposed with respect to the distal end 1101d of the first jaw 1101 and some regions of the switching pulley shaft AX1 and the switching pulley shaft AX2 overlap each other.

The switching pulley shafts AX1 and AX2 may be regions where at least one region of the firing wires 1110 is wound or comes into contact to organize and guide the path of the firing wire 1110 before entering the pulley shafts JX1, JX2, JX3, JX4, and JX5. A more detailed description of the arrangement of the switching pulley shafts AX1 and AX2 will be provided below.

As shown in FIG. 27, the first firing wire 1111 and the second firing wire 1112 may be correspondingly wound around the first fixed pulley 1121 and the second fixed pulley 1122 in the first jaw 1101 to be redirected, and connected to the rear of the end tool 1100 via at least one region of each of the switching pulley shafts AX1 and AX2 and the pulley shafts JX1, JX2, JX3, JX4, and JX5. Furthermore, the first firing wire 1111 and the second firing wire 1112 may further extend to a manipulation part 1200 via the connection part 1400 to be precisely controlled. Accordingly, precise motion control of the operation member 1140 may be easily implemented, and details thereof will be described later.

The jaw 1103 of the end tool 1100 will be described in more detail.

FIG. 28 is a schematic perspective view of the end tool of FIG. 27 with a cartridge removed.

The second jaw 1102 may be formed in an elongated bar shape as a whole, and for example, the second jaw 1102 may be formed in a rod shape to correspond to the first jaw 1101 in at least one region.

A proximal end 1102p of the second jaw 1102 may include a region that is coupled to the first jaw 1101. As an example, the proximal end 1102p is configured to be rotatable around one shaft JX of the proximal end 1102p with respect to the first jaw 1101.

The second jaw 1102 may have various forms, and as a specific example, a plurality of anvil grooves may be formed in at least one region of a surface of the second jaw 1102 facing the first jaw 1101, and the anvil groove may have a shape corresponding to the shape of the staple 1530.

The anvil groove of the second jaw 1102 may serve as a support for allowing the staple 1530 to be bent when the operation member 1140 pushes and raises the staple 1530 during a staple motion.

The second jaw 1102 includes a guide groove 1102a. The guide groove 1102a may have a shape elongated in a longitudinal direction of the second jaw 1102.

The guide groove 1102a may be formed to guide the operation member 1140, and may be a groove formed to pass through a region facing the operation member 1140. Through this, one region of the operation member 1140, such as at least one region of the body 1142 of the operation member 1140, or a first clamp 1146 connected thereto may pass through the guide groove 1102a to exit to the outside of the second jaw 1102. When the operation member 1140 moves forward, the first clamp 1146 may pass through the guide groove 1102a of the second jaw 1102 to be exposed to the outside of the second jaw 1102, and may come into contact with an upper surface of the second jaw 1102 or apply pressure thereto. As the operation member 1140 moves, the first clamp 1146 applies pressure on the upper surface of the second jaw 1102 and a second clamp 1147 to be described below applies pressure on a lower surface of the first jaw 1101 such that a gap between the second jaw 1102 and the first jaw 1101 decreases, allowing the second jaw 1102 to naturally remain in a closed state with respect to the first jaw 1101.

FIG. 29 is a transparent perspective view of FIG. 28. FIG. 30 is a perspective view illustrating the first jaw and the cartridge of the surgical instrument of FIG. 24.

Referring to FIGS. 29 and 30 and the like, the first jaw 1101 is formed in an elongated bar shape as a whole, and a rotational shaft may be disposed in the proximal end such that the first jaw 1101 is rotationally movable, and such a rotational shaft may correspond to the rotational shaft JX formed in the second jaw 1102 described above. In some embodiments, the cartridge 1500 may be accommodated closer to the distal end 1101d side than the rotational shaft.

For example, the first jaw 1101 may be formed entirely in the form of a hollow box with one surface (upper surface) thereof removed, such that a cartridge accommodation part 1101a capable of accommodating the cartridge 1500 may be formed inside the first jaw 1101. That is, the first jaw 1101 may be formed in a substantially “U”shape in cross section.

A guide groove 1101h may be formed in a bottom surface of the first jaw 1101, the bottom surface opposite to an upper open region formed by removing one surface. In some embodiments, the guide groove 1101h may be configured to guide a linear motion of the operation member 1140.

The guide groove 1101h may be configured to guide the operation member 1140, and may be a groove formed to pass through a region facing the operation member 1140. Through this, one region of the operation member 1140, such as at least one region of the body 1142 of the operation member 1140, or the second clamp 1147 connected thereto may pass through the guide groove 1101h to exit to the outside of the first jaw 1101. When the operation member 1140 moves forward, the second clamp 1147 may pass through the guide groove 1101h of the first jaw 1101 to be exposed to the outside of the first jaw 1101, and may come into contact with the lower surface of the first jaw 1101 or apply pressure thereto. As the operation member 1140 moves, the second clamp 1147 applies pressure on the lower surface of the first jaw 1101 and the first clamp 1146 applies pressure on the upper surface of the second jaw 1102 such that a gap between the second jaw 1102 and the first jaw 1101 decreases, allowing the second jaw 1102 to naturally remain in a closed state with respect to the first jaw 1101.

In an optional embodiment, the first jaw 1101 may include a window 1101b. After operating the operation member 1140 or using the end tool 1100, the second clamp 1147 of the operation member 1140 may be located corresponding to the window 1101b, and the coupled state of the first jaw 1101 and the operation member 1140 may be released.

The first jaw 1101 may include the front space 1101c located ahead of the cartridge accommodation part 1101a.

For example, the front space 1101c may be positioned closer to the distal end 1101d of the first jaw 1101 than the cartridge accommodation part 1101a. The plurality of fixed pulleys 1120 may be disposed in the front space 1101c, for example, the first fixed pulley 1121 and the second fixed pulley 1122 may be disposed in the front space 1101c.

According to an embodiment of the present disclosure, rotational resistance of pulleys in a surgical instrument can be minimized and space utilization of components can be improved.

Each of the embodiments described above is an embodiment that can be implemented independently, but it is also possible for the structures of each embodiment to be applied to other embodiments in a combined manner.

The present disclosure has been described above with a focus on exemplary embodiments. It will be understood by those skilled in the art that various changes in form and details may be made therein without departing from the essential features of the present disclosure. Therefore, the disclosed embodiments should be considered in descriptive sense only and not for purposes of limitation. The scope of the present disclosure is defined not by the detailed description of the disclosure but by the appended claims, and all differences within the scope will be construed as being included in the present disclosure.

What is claimed is:

1. An end tool for a surgical instrument, the end tool comprising:
one or more jaws configured to be rotatable;
a wire unit including a first wire and a second wire configured to transmit a driving force to the one or more jaws; and
a pulley unit positioned to contact with at least a portion of the wire unit and configured to guide a traveling path of the wire unit,
wherein the pulley unit includes at least one double-groove pulley including a first pulley part having a first groove and a second pulley part having a second groove, the second groove being configured to be spaced apart from the first groove, and
the first pulley part and the second pulley part are integrally formed.

2. The end tool of claim 1, wherein
the first wire is wound around the first groove, and
the second wire is wound around the second groove.

3. The end tool of claim 1, wherein
with respect to a rotational shaft of the at least one double-groove pulley,
the first wire is positioned on one side of the at least one double-groove pulley, and
the second wire is positioned on another side of the at least one double-groove pulley.

4. The end tool of claim 3, wherein, when the at least one double-groove pulley rotates, the first wire and the second wire, which are wound around the at least one double-groove pulley, move in different directions.

5. The end tool of claim 1, wherein the first wire and the second wire are positioned on same side of the at least one double-groove pulley with respect to a rotational shaft of the at least one double-groove pulley.

6. The end tool of claim 5, wherein, when the at least one double-groove pulley rotates, the first wire and the second wire, which are wound around the at least one double-groove pulley, move in a same direction.

7. The end tool of claim 1, wherein the first groove has a width and the second groove has a width, each defined in a direction parallel to a rotational shaft of the at least one double-groove pulley, the width of the first groove and the width of the second groove being different from each other.

8. The end tool of claim 1, wherein a diameter of the first pulley part and a diameter of the second pulley part are different from each other.

9. The end tool of claim 1, wherein
a pair of wires are wound around the at least one double-groove pulley, and
the pair of wires are configured to control one degree of freedom of the one or more jaws.

10. The end tool of claim 1, wherein
the one or more jaws include:
a first jaw configured to accommodate at least one region of an operation member movable in at least one direction; and
a second jaw facing the first jaw and configured to rotate relative to the first jaw, and
the first wire and the second wire are connected to the operation member to enable the operation member to move in the at least one direction.

11. The end tool of claim 10, wherein
the first wire is a firing wire that moves the operation member toward a distal end of the end tool, and
the second wire is a retraction wire that moves the operation member toward a proximal end of the end tool.

12. The end tool of claim 10, wherein
the pulley unit further includes a first double-groove pulley and a second double-groove pulley, each having rotational axis parallel to or coincident with a first rotational shaft,
the wire unit further includes a third wire and a fourth wire which are connected to the operation member,
the first wire and the second wire are wound around the first double-groove pulley, and
the third wire and the fourth wire are wound around the second double-groove pulley.

13. The end tool of claim 12, wherein
the first wire and the third wire are firing wires that move the operation member toward a distal end of the end tool, and
the second wire and the fourth wire are retraction wires that move the operation member toward a proximal end of the end tool.

14. The end tool of claim 1, wherein
the pulley unit further includes a first double-groove pulley and a second double-groove pulley, each having rotational axis parallel to or coincident with a first rotational shaft,
the wire unit further includes a third wire and a fourth wire which are configured to transmit a driving force to the one or more jaws,
the first wire and the second wire are wound around the first double-groove pulley, and
the third wire and the fourth wire are wound around the second double-groove pulley.

15. The end tool of claim 14, wherein the pulley unit further includes:
a third double-groove pulley and a fourth double-groove pulley which are configured to be rotatable around a third rotational shaft different from the first rotational shaft; and
a fifth double-groove pulley and a sixth double-groove pulley which are each configured to rotate around a fourth rotational shaft disposed parallel to the third rotational shaft.

16. The end tool of claim 15, wherein
the first wire and the second wire sequentially come into contact with the first double-groove pulley, the third double-groove pulley, and the fifth double-groove pulley while moving from a distal end toward a proximal end of the end tool, and
the third wire and the fourth wire sequentially come into contact with the second double-groove pulley, the fourth double-groove pulley, and the sixth double-groove pulley.

17. The end tool of claim 14, wherein
with respect to the first rotational shaft,
the first wire and the fourth wire are positioned on one side of the first double-groove pulley and one side of the second double-groove pulley, respectively, and
the second wire and the third wire are positioned on another side of the first double-groove pulley and another side of the second double-groove pulley, respectively.

18. A double-groove pulley applicable to a surgical instrument, the double-groove pulley comprising:
a first pulley part including a first groove around which a first wire is wound; and
a second pulley part including a second groove, which is configured to be spaced apart from the first groove and around which a second wire is wound,
wherein the first pulley part and the second pulley part are integrally formed.

19. The double-groove pulley of claim 18, wherein the first wire and the second wire are configured to control one degree of freedom of the surgical instrument.

20. The double-groove pulley of claim 18, wherein
with respect to a rotational shaft of the double-groove pulley,
the first wire is positioned on one side of the double-groove pulley, and
the second wire is positioned on another side of the double-groove pulley.
